SEQUENCE LISTING (203 sequences)

SEQ ID NO: 1 - protein, 15 aa, Artificial
  Synthetic
SSWHISHDSIGVLIR

SEQ ID NO: 2 - protein, 15 aa, Artificial
  Synthetic
SHCVLGHDGRSRCII

SEQ ID NO: 3 - protein, 15 aa, Artificial
  Synthetic
SNWHITHDSIGVLVR

SEQ ID NO: 4 - protein, 15 aa, Artificial
  Synthetic
SFWHVTHDSTGVLIR

SEQ ID NO: 5 - protein, 15 aa, Artificial
  Synthetic
SNFDISHDSIGVFVR

SEQ ID NO: 6 - protein, 15 aa, Artificial
  Synthetic
SSWHISHDDIGVFIR

SEQ ID NO: 7 - protein, 15 aa, Artificial
  Synthetic
SRFHITHDNVGVFIH

SEQ ID NO: 8 - protein, 15 aa, Artificial
  Synthetic
STWYSTHDNTGVFIN

SEQ ID NO: 9 - protein, 15 aa, Artificial
  Synthetic
SSFHISHDNIGVFVR

SEQ ID NO: 10 - protein, 15 aa, Artificial
  Synthetic
SYFHVTHDSNGVFIH

SEQ ID NO: 11 - protein, 15 aa, Artificial
  Synthetic
SNLHITHDSTGVFIH

SEQ ID NO: 12 - protein, 15 aa, Artificial
  Synthetic
STFHSRHDNIGVFMS

SEQ ID NO: 13 - protein, 15 aa, Artificial
  Synthetic
SFFQSTHDNTGVFIR

SEQ ID NO: 14 - protein, 15 aa, Artificial
  Synthetic
SNWHSTHDSIGVFIS

SEQ ID NO: 15 - protein, 15 aa, Artificial
  Synthetic
SSFDSIHDSIGVFIR

SEQ ID NO: 16 - protein, 15 aa, Artificial
  Synthetic
SHLHSRHDNIGVFIH

SEQ ID NO: 17 - protein, 15 aa, Artificial
  Synthetic
SNFHSTHDSIGVFVS

SEQ ID NO: 18 - protein, 15 aa, Artificial
  Synthetic
STLNITHDTIGVFVS

SEQ ID NO: 19 - protein, 15 aa, Artificial
  Synthetic
STWHSSHDSIGVFIH

SEQ ID NO: 20 - protein, 15 aa, Artificial
  Synthetic
SSFHVTHDDVGVFLR

SEQ ID NO: 21 - protein, 15 aa, Artificial
  Synthetic
SAFHSSHDSIGVFIN

SEQ ID NO: 22 - protein, 15 aa, Artificial
  Synthetic
SSLYSTHDSIGVFVS

SEQ ID NO: 23 - protein, 15 aa, Artificial
  Synthetic
SSWHTTHDNTGVFIR

SEQ ID NO: 24 - protein, 15 aa, Artificial
  Synthetic
STLNSIHDSIGVFIN

SEQ ID NO: 25 - protein, 15 aa, Artificial
  Synthetic
STLHISHDSIGVLVR

SEQ ID NO: 26 - protein, 15 aa, Artificial
  Synthetic
SGWNITHDSIGVFMS

SEQ ID NO: 27 - protein, 15 aa, Artificial
  Synthetic
SSFHISHDAIGVFIN

SEQ ID NO: 28 - protein, 15 aa, Artificial
  Synthetic
SSWTITHDSIGVFMN

SEQ ID NO: 29 - protein, 15 aa, Artificial
  Synthetic
SNWTSSHDDIGVFLR

SEQ ID NO: 30 - protein, 15 aa, Artificial
  Synthetic
SSFHSVHDNIGVFVS

SEQ ID NO: 31 - protein, 15 aa, Artificial
  Synthetic
SSFHISHDSIGVFIN

SEQ ID NO: 32 - protein, 15 aa, Artificial
  Synthetic
STFHTTHDSTGVFIR

SEQ ID NO: 33 - protein, 15 aa, Artificial
  Synthetic
SPFHSSHDSIGVFVK

SEQ ID NO: 34 - protein, 15 aa, Artificial
  Synthetic
STWQRIHDDIGVFIS

SEQ ID NO: 35 - protein, 15 aa, Artificial
  Synthetic
STWQRIHDDIGVFIS

SEQ ID NO: 36 - protein, 15 aa, Artificial
  Synthetic
SSFHTTHDNIGVFIN

SEQ ID NO: 37 - protein, 15 aa, Artificial
  Synthetic
SPFHSSHDSIGVFVK

SEQ ID NO: 38 - protein, 15 aa, Artificial
  Synthetic
SPFHSSHDSIGVFVK

SEQ ID NO: 39 - protein, 15 aa, Artificial
  Synthetic
SIFHTTHDNTGVFIR

SEQ ID NO: 40 - protein, 15 aa, Artificial
  Synthetic
SKFVTTHDNIGVFMS

SEQ ID NO: 41 - protein, 15 aa, Artificial
  Synthetic
SKFVTTHDNIGVFMS

SEQ ID NO: 42 - protein, 15 aa, Artificial
  Synthetic
SRFHITHDDIGVFTY

SEQ ID NO: 43 - protein, 15 aa, Artificial
  Synthetic
SNWDSSHDSIGVFMR

SEQ ID NO: 44 - protein, 15 aa, Artificial
  Synthetic
STFHSRHDTIGVFFS

SEQ ID NO: 45 - protein, 15 aa, Artificial
  Synthetic
SSWHSNHDQVGVFIN

SEQ ID NO: 46 - protein, 15 aa, Artificial
  Synthetic
SSLHSKHDSVGVFIS

SEQ ID NO: 47 - protein, 15 aa, Artificial
  Synthetic
SNLRSSHDSIGVFMH

SEQ ID NO: 48 - protein, 15 aa, Artificial
  Synthetic
STFHNIHDSIGVFIN

SEQ ID NO: 49 - protein, 15 aa, Artificial
  Synthetic
SSWQISHDSTGVFIS

SEQ ID NO: 50 - protein, 14 aa, Artificial
  Synthetic
XXXXXHDXXGVXXX

SEQ ID NO: 51 - protein, 14 aa, Artificial
  Synthetic
XXXXXHXXXGXXXX

SEQ ID NO: 52 - protein, 14 aa, Artificial
  Synthetic
XXHXXHDXIGVXXX

SEQ ID NO: 53 - protein, 14 aa, Artificial
  Synthetic
XXHXXHDXXGVXXX

SEQ ID NO: 54 - protein, 11 aa, Artificial
  Synthetic
SIWDDFVGWSR

SEQ ID NO: 55 - protein, 11 aa, Artificial
  Synthetic
SIWDDWLGYSR

SEQ ID NO: 56 - protein, 11 aa, Artificial
  Synthetic
SIWDDWIGFSR

SEQ ID NO: 57 - protein, 10 aa, Artificial
  Synthetic
SIWDDFRASR

SEQ ID NO: 58 - protein, 18 aa, Artificial
  Synthetic
SIWDDYIGWTASGVGTSR

SEQ ID NO: 59 - protein, 18 aa, Artificial
  Synthetic
SIWDDYNRWLRGNSDISR

SEQ ID NO: 60 - protein, 18 aa, Artificial
  Synthetic
SIWDDYTRWGHKEASSSR

SEQ ID NO: 61 - protein, 18 aa, Artificial
  Synthetic
SIWHDFKSWKDNTPYHSR

SEQ ID NO: 62 - protein, 18 aa, Artificial
  Synthetic
SIWSDYLKWNAARGGASR

SEQ ID NO: 63 - protein, 18 aa, Artificial
  Synthetic
SIYDDFLNWKHGSVVPSR

SEQ ID NO: 64 - protein, 18 aa, Artificial
  Synthetic
SIYDDFRNWQISRVSDSR

SEQ ID NO: 65 - protein, 18 aa, Artificial
  Synthetic
SIYDDFVRWAMSSRADSR

SEQ ID NO: 66 - protein, 18 aa, Artificial
  Synthetic
SIYDNYLKWQERDRVTSR

SEQ ID NO: 67 - protein, 17 aa, Artificial
  Synthetic
SIWADYVRSSSASPLSR

SEQ ID NO: 68 - protein, 11 aa, Artificial
  Synthetic
SAWDDWLGYSR

SEQ ID NO: 69 - protein, 11 aa, Artificial
  Synthetic
SDWDDWLGYSR

SEQ ID NO: 70 - protein, 11 aa, Artificial
  Synthetic
SEWDDWLGYSR

SEQ ID NO: 71 - protein, 11 aa, Artificial
  Synthetic
SFWDDWLGYSR

SEQ ID NO: 72 - protein, 11 aa, Artificial
  Synthetic
SGWDDWLGYSR

SEQ ID NO: 73 - protein, 11 aa, Artificial
  Synthetic
SHWDDWMGYSR

SEQ ID NO: 74 - protein, 11 aa, Artificial
  Synthetic
SIWDDWLGYSR

SEQ ID NO: 75 - protein, 11 aa, Artificial
  Synthetic
SKWDDWLGYSR

SEQ ID NO: 76 - protein, 11 aa, Artificial
  Synthetic
SLWDDWLGYSR

SEQ ID NO: 77 - protein, 11 aa, Artificial
  Synthetic
SMWDDWLGYSR

SEQ ID NO: 78 - protein, 11 aa, Artificial
  Synthetic
SNWDDWLGYSR

SEQ ID NO: 79 - protein, 11 aa, Artificial
  Synthetic
SPWDDWLGYSR

SEQ ID NO: 80 - protein, 11 aa, Artificial
  Synthetic
SRWDDWLGYSR

SEQ ID NO: 81 - protein, 11 aa, Artificial
  Synthetic
SSWDDWLGYSR

SEQ ID NO: 82 - protein, 11 aa, Artificial
  Synthetic
STWDDWLGYSR

SEQ ID NO: 83 - protein, 11 aa, Artificial
  Synthetic
SVWDDWLGYSR

SEQ ID NO: 84 - protein, 11 aa, Artificial
  Synthetic
SYWDDWLGYSR

SEQ ID NO: 85 - protein, 11 aa, Artificial
  Synthetic
SICDDWLGYSR

SEQ ID NO: 86 - protein, 11 aa, Artificial
  Synthetic
SIFDDWLGYSR

SEQ ID NO: 87 - protein, 11 aa, Artificial
  Synthetic
SIGDDWLGYSR

SEQ ID NO: 88 - protein, 11 aa, Artificial
  Synthetic
SIIDDWLGYSR

SEQ ID NO: 89 - protein, 11 aa, Artificial
  Synthetic
SIKDDWLGYSR

SEQ ID NO: 90 - protein, 11 aa, Artificial
  Synthetic
SILDDWLGYSR

SEQ ID NO: 91 - protein, 11 aa, Artificial
  Synthetic
SIMDDWLGYSR

SEQ ID NO: 92 - protein, 11 aa, Artificial
  Synthetic
SINDDWLGYSR

SEQ ID NO: 93 - protein, 11 aa, Artificial
  Synthetic
SIPDDWLGYSR

SEQ ID NO: 94 - protein, 11 aa, Artificial
  Synthetic
SIQDDWLGYSR

SEQ ID NO: 95 - protein, 11 aa, Artificial
  Synthetic
SISDDWLGYSR

SEQ ID NO: 96 - protein, 11 aa, Artificial
  Synthetic
SIVDDWLGYSR

SEQ ID NO: 97 - protein, 11 aa, Artificial
  Synthetic
SIWADWLGYSR

SEQ ID NO: 98 - protein, 11 aa, Artificial
  Synthetic
SIWEDWLGYSR

SEQ ID NO: 99 - protein, 11 aa, Artificial
  Synthetic
SIWFDWLGYSR

SEQ ID NO: 100 - protein, 11 aa, Artificial
  Synthetic
SIWGDWLGYSR

SEQ ID NO: 101 - protein, 11 aa, Artificial
  Synthetic
SIWHDWLGYSR

SEQ ID NO: 102 - protein, 11 aa, Artificial
  Synthetic
SIWIDWLGYSR

SEQ ID NO: 103 - protein, 11 aa, Artificial
  Synthetic
SIWKDWLGYSR

SEQ ID NO: 104 - protein, 11 aa, Artificial
  Synthetic
SIWLDWLGYSR

SEQ ID NO: 105 - protein, 11 aa, Artificial
  Synthetic
SIWMDWLGYSR

SEQ ID NO: 106 - protein, 11 aa, Artificial
  Synthetic
SIWNDWLGYSR

SEQ ID NO: 107 - protein, 11 aa, Artificial
  Synthetic
SIWPDWLGYSR

SEQ ID NO: 108 - protein, 11 aa, Artificial
  Synthetic
SIWQDWLGYSR

SEQ ID NO: 109 - protein, 11 aa, Artificial
  Synthetic
SIWRDWLGYSR

SEQ ID NO: 110 - protein, 11 aa, Artificial
  Synthetic
SIWSDWLGYSR

SEQ ID NO: 111 - protein, 11 aa, Artificial
  Synthetic
SIWTDWLGYSR

SEQ ID NO: 112 - protein, 11 aa, Artificial
  Synthetic
SIWVDWLGYSR

SEQ ID NO: 113 - protein, 11 aa, Artificial
  Synthetic
SIWWDWLGYSR

SEQ ID NO: 114 - protein, 11 aa, Artificial
  Synthetic
SIWYDWLGYSR

SEQ ID NO: 115 - protein, 11 aa, Artificial
  Synthetic
SIWDFWLGYSR

SEQ ID NO: 116 - protein, 11 aa, Artificial
  Synthetic
SIWDGWLGYSR

SEQ ID NO: 117 - protein, 11 aa, Artificial
  Synthetic
SIWDHWLGYSR

SEQ ID NO: 118 - protein, 11 aa, Artificial
  Synthetic
SIWDKWLGYSR

SEQ ID NO: 119 - protein, 11 aa, Artificial
  Synthetic
SIWDLWLGYSR

SEQ ID NO: 120 - protein, 11 aa, Artificial
  Synthetic
SIWDMWLGYSR

SEQ ID NO: 121 - protein, 11 aa, Artificial
  Synthetic
SIWDNWLGYSR

SEQ ID NO: 122 - protein, 11 aa, Artificial
  Synthetic
SIWDPWLGYSR

SEQ ID NO: 123 - protein, 11 aa, Artificial
  Synthetic
SIWDQWLGYSR

SEQ ID NO: 124 - protein, 11 aa, Artificial
  Synthetic
SIWDRWLGYSR

SEQ ID NO: 125 - protein, 11 aa, Artificial
  Synthetic
SIWDSWLGYSR

SEQ ID NO: 126 - protein, 11 aa, Artificial
  Synthetic
SIWDTWLGYSR

SEQ ID NO: 127 - protein, 11 aa, Artificial
  Synthetic
SIWDVWLGYSR

SEQ ID NO: 128 - protein, 11 aa, Artificial
  Synthetic
SIWDWWLGYSR

SEQ ID NO: 129 - protein, 11 aa, Artificial
  Synthetic
SIWDYWLGYSR

SEQ ID NO: 130 - protein, 11 aa, Artificial
  Synthetic
SIWDDALGYSR

SEQ ID NO: 131 - protein, 11 aa, Artificial
  Synthetic
SIWDDCLGYSR

SEQ ID NO: 132 - protein, 11 aa, Artificial
  Synthetic
SIWDDDLGYSR

SEQ ID NO: 133 - protein, 11 aa, Artificial
  Synthetic
SIWDDELGYSR

SEQ ID NO: 134 - protein, 11 aa, Artificial
  Synthetic
SIWDDFLGYSR

SEQ ID NO: 135 - protein, 11 aa, Artificial
  Synthetic
SIWDDGLGYSR

SEQ ID NO: 136 - protein, 11 aa, Artificial
  Synthetic
SIWDDHLGYSR

SEQ ID NO: 137 - protein, 11 aa, Artificial
  Synthetic
SIWDDILGYSR

SEQ ID NO: 138 - protein, 11 aa, Artificial
  Synthetic
SIWDDKLGYSR

SEQ ID NO: 139 - protein, 11 aa, Artificial
  Synthetic
SIWDDLLGYSR

SEQ ID NO: 140 - protein, 11 aa, Artificial
  Synthetic
SIWDDNLGYSR

SEQ ID NO: 141 - protein, 11 aa, Artificial
  Synthetic
SIWDDRLGHSR

SEQ ID NO: 142 - protein, 11 aa, Artificial
  Synthetic
SIWDDSLGYSR

SEQ ID NO: 143 - protein, 11 aa, Artificial
  Synthetic
SIWDDWAGYSR

SEQ ID NO: 144 - protein, 11 aa, Artificial
  Synthetic
SIWDDWCGYSR

SEQ ID NO: 145 - protein, 11 aa, Artificial
  Synthetic
SIWDDWFGYSR

SEQ ID NO: 146 - protein, 11 aa, Artificial
  Synthetic
SIWDDWGGYSR

SEQ ID NO: 147 - protein, 11 aa, Artificial
  Synthetic
SIWDDWHGYSR

SEQ ID NO: 148 - protein, 11 aa, Artificial
  Synthetic
SIWDDWIGYSR

SEQ ID NO: 149 - protein, 11 aa, Artificial
  Synthetic
SIWDDWKGYSR

SEQ ID NO: 150 - protein, 11 aa, Artificial
  Synthetic
SIWDDWPGYSR

SEQ ID NO: 151 - protein, 11 aa, Artificial
  Synthetic
SIWDDWQGYSR

SEQ ID NO: 152 - protein, 11 aa, Artificial
  Synthetic
SIWDDWRGYSR

SEQ ID NO: 153 - protein, 11 aa, Artificial
  Synthetic
SIWDDWSGYSR

SEQ ID NO: 154 - protein, 11 aa, Artificial
  Synthetic
SIWDDWTGYSR

SEQ ID NO: 155 - protein, 11 aa, Artificial
  Synthetic
SIWDDWVGYSR

SEQ ID NO: 156 - protein, 11 aa, Artificial
  Synthetic
SIWDDWYGYSR

SEQ ID NO: 157 - protein, 11 aa, Artificial
  Synthetic
SIWDDWLCYSR

SEQ ID NO: 158 - protein, 11 aa, Artificial
  Synthetic
SIWDDWLDYSR

SEQ ID NO: 159 - protein, 11 aa, Artificial
  Synthetic
SIWDDWLFYSR

SEQ ID NO: 160 - protein, 11 aa, Artificial
  Synthetic
SIWDDWLHYSR

SEQ ID NO: 161 - protein, 11 aa, Artificial
  Synthetic
SIWDDWLIYSR

SEQ ID NO: 162 - protein, 11 aa, Artificial
  Synthetic
SIWDDWLKYSR

SEQ ID NO: 163 - protein, 11 aa, Artificial
  Synthetic
SIWDDWLLYSR

SEQ ID NO: 164 - protein, 11 aa, Artificial
  Synthetic
SIWDDWLMYSR

SEQ ID NO: 165 - protein, 11 aa, Artificial
  Synthetic
SIWDDWLPYSR

SEQ ID NO: 166 - protein, 11 aa, Artificial
  Synthetic
SIWDDWLQYSR

SEQ ID NO: 167 - protein, 11 aa, Artificial
  Synthetic
SIWDDWLRYSR

SEQ ID NO: 168 - protein, 11 aa, Artificial
  Synthetic
SIWDDWLSYSR

SEQ ID NO: 169 - protein, 11 aa, Artificial
  Synthetic
SIWDDWLVYSR

SEQ ID NO: 170 - protein, 11 aa, Artificial
  Synthetic
SIWDDWLYYSR

SEQ ID NO: 171 - protein, 11 aa, Artificial
  Synthetic
SIWDDWLGASR

SEQ ID NO: 172 - protein, 11 aa, Artificial
  Synthetic
SIWDDWLGESR

SEQ ID NO: 173 - protein, 11 aa, Artificial
  Synthetic
SIWDDWLGFSR

SEQ ID NO: 174 - protein, 11 aa, Artificial
  Synthetic
SIWDDWLGGSR

SEQ ID NO: 175 - protein, 11 aa, Artificial
  Synthetic
SIWDDWLGHSR

SEQ ID NO: 176 - protein, 11 aa, Artificial
  Synthetic
SIWDDWLGISR

SEQ ID NO: 177 - protein, 11 aa, Artificial
  Synthetic
SIWDDWLGLSR

SEQ ID NO: 178 - protein, 11 aa, Artificial
  Synthetic
SIWDDWLGMSR

SEQ ID NO: 179 - protein, 11 aa, Artificial
  Synthetic
SIWDDWLGNSR

SEQ ID NO: 180 - protein, 11 aa, Artificial
  Synthetic
SIWDDWLGPSR

SEQ ID NO: 181 - protein, 11 aa, Artificial
  Synthetic
SIWDDWLGRSR

SEQ ID NO: 182 - protein, 11 aa, Artificial
  Synthetic
SIWDDWLGSSR

SEQ ID NO: 183 - protein, 11 aa, Artificial
  Synthetic
SIWDDWLGTSR

SEQ ID NO: 184 - protein, 11 aa, Artificial
  Synthetic
SIWDDWLGVSR

SEQ ID NO: 185 - protein, 10 aa, Artificial
  Synthetic
IWDDWIGYSR

SEQ ID NO: 186 - protein, 9 aa, Artificial
  Synthetic
WDDWIGYSR

SEQ ID NO: 187 - protein, 8 aa, Artificial
  Synthetic
DDWIGYSR

SEQ ID NO: 188 - protein, 7 aa, Artificial
  Synthetic
DWIGYSR

SEQ ID NO: 189 - protein, 10 aa, Artificial
  Synthetic
SIWDDWIGYS

SEQ ID NO: 190 - protein, 9 aa, Artificial
  Synthetic
SIWDDWIGY

SEQ ID NO: 191 - protein, 8 aa, Artificial
  Synthetic
SIWDDWIG

SEQ ID NO: 192 - protein, 7 aa, Artificial
  Synthetic
SIWDDWI

SEQ ID NO: 193 - protein, 9 aa, Artificial
  Synthetic
IWDDWIGYS

SEQ ID NO: 194 - protein, 8 aa, Artificial
  Synthetic
IWDDWIGY

SEQ ID NO: 195 - protein, 7 aa, Artificial
  Synthetic
WDDWIGY

SEQ ID NO: 196 - protein, 6 aa, Artificial
  Synthetic
WDDWIG

SEQ ID NO: 197 - protein, 6 aa, Artificial
  Synthetic
IWDDWI

SEQ ID NO: 198 - protein, 9 aa, Artificial
  Synthetic
SIWDDXXXY

SEQ ID NO: 199 - protein, 9 aa, Artificial
  Synthetic
SIXDDXXXW

SEQ ID NO: 200 - protein, 9 aa, Artificial
  Synthetic
SIXDDXXXX

SEQ ID NO: 201 - protein, 9 aa, Artificial
  Synthetic
XIXDDXVRX

SEQ ID NO: 202 - protein, 9 aa, Artificial
  Synthetic
SIXXDXXXX

SEQ ID NO: 203 - protein, 9 aa, Artificial
  Synthetic
SIXXDXXXX

(19) United States
(12) Patent Application Publication
(10) Pub. No.: US 2014/0079753 A1
(43) Pub. Date: Mar. 20, 2014

(54) BMP BINDING PEPTIDES

(71) Applicant: Affinergy, LLC, Durham, NC (US)

(72) Inventors: Martyn Kerry Darby, Chapel Hill, NC (US); Dalia Isolda Juzumiene, Cary, NC (US); Isaac Gilliam Sanford, Durham, NC (US); Jonathan Allen Hodges, Durham, NC (US); Shrikumar Ambujakshan Nair, Cary, NC (US)

(73) Assignee: Affinergy, LLC, Durham, NC (US)

(21) Appl. No.: 14/082,347

(22) Filed: Nov. 18, 2013

Related U.S. Application Data
(63) Continuation of application No. PCT/US2011/037064, filed on May 18, 2011.

(51) Int. Cl. C07K 7/06; A61K 38/08; G01N 33/74; A61K 47/48

(52) U.S. Cl. CPC C07K 7/06; A61K 47/48292; A61K 38/08; G01N 33/74
USPC 424/423; 530/300; 514/8.8; 530/356; 424/602; 435/7.1; 436/501

(57) ABSTRACT

Compositions and methods for tissue repair are provided including cell binding peptides and BMP binding peptides. The cell binding peptides bind to one or more of stem cells and fibroblasts. The tissue for repair includes bone, tendon, muscle, connective tissue, ligament, cardiac tissue, bladder tissue, or dermis. Implantable devices for tissue repair are provided to which the cell and/or BMP binding peptides are attached, such as acellular extracellular matrix having attached binding peptide and bone graft material comprising a ceramic.

### CROSS REFERENCE TO RELATED APPLICATIONS

This application is a continuation of International Patent Application No. PCT/US2011/037064 filed May 18, 2011, the disclosure of which is hereby incorporated by reference in its entirety.

### GRANT STATEMENT

The invention was made with government support under Grant No. 5R44DE020760-03 and Grant No. 5R44DE018071-03 awarded by the National Institute of Dental and Craniofacial Research; and under Grant No. 5R44GM077753-03, Grant No. 3R44GM083380-02, and Grant No. 1R43GM093462-01 awarded by the National Institute of General Medical Sciences. The government has certain rights in the invention.

### FIELD

The presently disclosed subject matter relates to the capture of cells and BMPs onto implantable devices for tissue repair.

### BACKGROUND

Multipotent stem cells are known to play a role in healing and repair in response to trauma, disease or disorder. Stem cell mediated repair and healing are achieved by proliferation and differentiation of the stem cells into specialized cell types. For example, mesenchymal stem cells can differentiate into cell types such as bone, cartilage, fat, ligament, muscle, and tendon. In the case of defects in bone, mesenchymal stem cells from the bone marrow, periosteum, and surrounding soft tissue proliferate and differentiate into specialized bone cells. Stem cells can be obtained from embryonic or adult tissues of humans or other animals. As a result of the healing activity of stem cells, much focus has been placed on using stem cells as a treatment to aid in the remodeling of damaged tissue into healthy tissue.

In addition to stem cells, fibroblast cells have a role in soft tissue repair. Hernia repair is one of the most common surgical procedures world-wide, with over 20 million repairs performed each year (Kingsnorth, A. and K. LeBlanc, Lancet, 2003, 362:1561-71). In the US there are approximately 100,000 incisional hernia repairs performed annually costing an estimated 1.7 billion dollars (Finan et al., Hernia, 2009, 13:173-82). Despite advances, recurrence rates remain high and range from 3-60% with an average rate of 25% for an initial repair and 44% after a second repair (Afifi, R. Y., Hernia, 2005, 9:310-5; Gray et al., Am J Surg, 2008, 196:201-6). Biocompatible materials have triggered a rapid evolution of hernia repair techniques over the past 10 years. High-tension fascial suturing to strengthen the abdominal wall has been replaced by low-tension repair using biocompatible synthetic mesh (Luijendijk et al., N Engl J Med, 2000, 343:392-98; Flum et al., Ann Surg, 2003, 237:129-35). While a modest improvement over basic suturing, synthetic mesh harbors all the potential pitfalls of implanting a permanent foreign body: adhesions, potential infection, chronic pain, and subsequent mesh removal (Flum et al., Ann Surg, 2003, 237:129-35; Conze et al., Langenbecks Arch Surg, 2007. 392:453-37). Allograft and xenograft materials such as, for example, acellular dermal matrix (ADM) and porcine small intestine submucosa have emerged as favorable alternatives to synthetics, especially in patients with comorbidities, for many types of soft tissue repair including wound, abdominal wall, tendon, breast, dura matter, and rotator cuff repair (Diaz et al., Am Surg, 2006, 72:1181-88; Kim et al., Am J Surg, 2006, 192:705-9; Kish et al., Am Surg, 2005, 71:1047-50; Butler, C. E., Clin Plastic Surg, 2006, 33:199-211; Badylak, S. F., Biomaterials, 2007, 28:3587-93; Longo et al., British Medical Bulletin, 2010, 94:165-88), maintaining an intact elastin lattice, as well as channels for capillary microvascularization. These collagen-based materials promote key components of wound healing and are bioabsorbable. However, complication rates of 24% with recurrence being the most common complication have been reported with these materials, and design improvements are needed (Gupta, A., et al., Hernia, 2006, 10:419-25; Misra, S., et al., Hernia, 2008, 12:247-50). Wound breaking strength represents the amount of force a surgical wound can withstand before failing, and failure occurs when there is a deficient quantity and quality of tissue repair (Franz, M. G., Surg Clin North Am, 2008, 88:1-15, vii). Previous studies have suggested that wound repair integrity reaches a normal breaking strength in 30 days (Franz et al., J Surg Res, 2001, 97: 109-16; Robson, M. C., Surg Clin North Am, 2003, 83:557-69). Fibroblasts are responsible for collagen synthesis and deposition and recovery of wound breaking strength (Franz, M. G., Surg Clin North Am, 2008, 88:1-15, vii). Two days post surgery the inflammatory response subsides and fibroblasts infiltrate the wound, out numbering other cell types by day 4 (Dubay, D. A. and M. G. Franz, Surg Clin North Am, 2003, 83:463-81). Wounds are increasingly challenged during the recovery period as patients return to normal activity. Therefore, a medical device that can become populated with fibroblasts and vascularize faster than other bioprosthetics would reduce the recovery time and increase healing rates to improve repair outcomes.

In addition to cells, certain BMPs have a role in healing and repair in response to trauma, disease or disorder. In particular, the bone morphogenic proteins (BMP), including BMP-2 and BMP-7, have shown clinical benefit in the treatment of bone fractures and spine fusions. Back pain is one of the leading reasons for physician visits in the United States and in many cases requires surgical intervention. In 2009 in the United States, there were 425,000 spinal fusion surgeries, and the frequency of these surgeries is projected to grow 6% per year. The gold standard for bone graft in spinal fusion is autograft from the iliac crest; however, the use of autograft presents multiple challenges including donor site morbidity, blood loss, limited availability, prolonged operating times, and pseudarthrosis due to a slow rate of fusion. Bone marrow aspirate (BMA) contains osteoinductive factors and can be harvested at point-of-care without the complications of harvesting autogenous bone. However, the current bone graft substitutes are not adequate for the retention and release of osteoinductive factors from BMA over the length of the healing cycle. As a result, there is a large effort to develop bone graft substitutes or extenders that can not only reduce or replace the need for harvest of autogenous bone but also accelerate the rate of fusion (arthrodesis). Tricalcium phosphate (TCP)-based bone graft substitutes often containing collagen are used commonly in lumbar spinal fusion because TCP is resorbed over several months as bone heals. Ceramic bone graft substitutes, such as the MASTERGRAFT and VITOSS line of products, have been used successfully in spinal fusion surgeries (Miyazaki et al., Eur Spine J, 2009, 18:783-99; Khan et al., Am Acad Orthop Surg, 2005, 13:129-37; Neen et al., Spine, 2006, 31:E636-40; Epstein, Spine J, 2009, 9:630-8; Carter, Spine J, 2009, 9:434-8; Epstein, J Spinal Disord Tech, 2006, 19:424-9; Birch, N. and W. L. D'Souza, J Spinal Disord Tech, 2009, 22:434-8; Lerner, T., V., Eur Spine J, 2009, 18:170-9; Knop. et al., Arch Orthop Trauma Surg, 2006, 126:204-10; Epstein, Spine J, 2008, 8:882-7), in particular when used in combination with the recombinant BMP-2-containing product INFUSE (Glassman et al., Spine J, 2007, 7:44-9; Boden et al., Spine, 2002, 27:2662-73; Glassman, Spine, 2005, 30:1694-8). Recombinant BMP-2 is effective but carries a high cost and serious safety risks (Cahill et al., JAMA, 2009, 302:58-66), in part because of leakage away from its carrier and the high dose required to achieve therapeutic levels (Poynton, A. R. and J. M. Lane, Spine, 2002, 27:S40-8). Accordingly, there remains an unmet clinical need in bone repair and spinal fusion surgery for a safe, cost-effective bone graft substitute that can provide a sustained dose of osteoinductive factors for the healing process.

Therefore, while tissue remodeling can theoretically be achieved by application of cells and/or BMPs at the site of damaged tissue, several obstacles stand in the way of this regenerative technology becoming reality. One obstacle is that cells and/or BMPs injected into many tissues are rapidly cleared via the lymphatics or vascular drainage. In addition, the exogenous BMPs can have undesirable ectopic effects. Another obstacle is that the most widely used source of stem cells, bone marrow aspirate, often provides an inadequate amount of stem cells. As a result, use of allogeneic stem cells or culturing of stem cells to increase their number prior to use is frequently still required. The presently disclosed subject matter provides systems for locally binding, delivering, and retaining cells and BMPs at the site of tissues in need of healing or repair.

### SUMMARY

The presently disclosed subject matter provides compositions and methods for tissue repair including cell- and BMP-binding peptides and implantable devices for tissue repair comprising the attached binding peptides. In one embodiment, the presently disclosed subject matter provides an implantable device for tissue repair comprising a biopolymer having a covalently attached cell binding peptide and/or BMP binding peptide.

In one embodiment, the presently disclosed subject matter provides a method for tissue repair, comprising: delivering to a subject an implantable device for tissue repair, wherein the implantable device comprises a biopolymer having a covalently attached cell binding peptide and/or BMP binding peptide, and wherein the implantable device serves as a scaffold for tissue repair. In one embodiment, the tissue for repair is a soft tissue comprising any one or more of tendon, muscle, connective tissue, ligament, cardiac tissue, bladder tissue, or dermis. In one embodiment, the tissue for repair is a bone tissue, and the implantable device comprising the biopolymer is a bone graft material comprising a ceramic.

In one embodiment, the presently disclosed subject matter provides a method for capturing cells and/or BMP onto an implantable device for tissue repair, comprising: contacting a sample comprising cells and/or BMP with the implantable device, wherein the implantable device comprises a biopolymer having a covalently attached cell binding peptide and/or BMP binding peptide, wherein the cells and/or BMP comprised in the sample are captured onto the implantable device through binding to the attached binding peptide.

In one embodiment, the presently disclosed subject matter provides a method for tissue repair, comprising: contacting a sample comprising cells and/or BMP with an implantable device comprising a biopolymer having a covalently attached cell binding peptide and/or BMP binding peptide, wherein the cells and/or BMP comprised in the sample are captured onto the implantable device through binding to the attached binding peptide; and delivering to a subject the implantable device for the tissue repair comprising the captured cells and/or BMP, wherein the presence of the captured cells and/or BMP promotes tissue growth in the subject.

In one embodiment, the presently disclosed subject matter provides a method for capturing cells, comprising contacting a sample comprising cells with a cell binding peptide attached to a substrate, wherein the cells comprised in the sample are captured onto the substrate through binding to the cell binding peptide. In one embodiment, the method comprises a step of releasing the captured cells from the substrate, wherein the step of releasing the captured stem cells is one or more of a physical means, chemical means, or photoactivated means. In one embodiment, the released cells are delivered to a subject. In one embodiment, the presently disclosed subject matter provides a device for chromatography comprising a cell binding peptide attached to a substrate.

In one embodiment, the presently disclosed subject matter provides a method for visualizing cells, comprising contacting a cell with a cell binding peptide comprising a visualization agent, wherein the cell binding peptide binds to the cell to enable cell visualization.

### BRIEF DESCRIPTION OF THE DRAWINGS

FIG. 1 is a schematic diagram depicting one method for covalently attaching a binding peptide to a substrate comprising amino functional groups.

FIG. 2 is a schematic diagram depicting one method for covalently attaching a binding peptide to a substrate comprising amino functional groups.

FIG. 3 is a schematic diagram depicting methods for covalently attaching a binding peptide to a substrate having an amino functional group.

FIG. 4 is a schematic diagram depicting one method for covalently attaching a binding peptide to a substrate comprising amino functional groups.

FIG. 5 is a schematic diagram depicting one method for covalently attaching a binding peptide to a substrate comprising amino functional groups.

FIG. 6 FIG. 7 is a schematic diagram depicting exemplary chemistry for covalently attaching a binding peptide to chitosan.

FIG. 8 is a schematic diagram depicting exemplary chemistry for covalently attaching a binding peptide to chitosan.

FIG. 9 is a schematic diagram depicting exemplary chemistry for covalently attaching a binding peptide to hyaluronic acid.

FIG. 10 is a schematic diagram depicting exemplary chemistry for covalently attaching a binding peptide to hyaluronic acid.

FIG. 11 is a schematic diagram depicting exemplary chemistry for introducing an amino functional group on cellulose for subsequent covalent attachment of a binding peptide.

FIG. 12 is a schematic diagram depicting exemplary chemistry for covalently attaching a binding peptide to oxidized cellulose.

FIG. 13 is a schematic diagram depicting one method for covalently attaching more than one binding peptide to a substrate comprising amino functional groups.

FIG. 14 is a table showing an alignment of phage peptide sequences resulting from a mutagenesis study of cell binding peptide SEQ ID NO: 1. The single letter amino acid sequence of SEQ ID NO: 1 is shown at the top in white letters with black shading. The phage that retained cell binding activity to adipose derived stem cells (ASCs) and fibroblasts are listed below SEQ ID NO: 1 with original amino acids in white with black shading. Amino acid substitutions are shown in black letters with white shading. The phage from the mutagenesis that did not exhibit cell binding activity are not shown.

FIG. 15 is a bar graph showing the ability of cell binding peptide SEQ ID NOs: 1 & 2 to specifically bind human adipose-derived mesenchymal stem cells (hASC) compared to a number of other cells types including rabbit adipose-derived mesenchymal stem cells (Rabbit ASC), rat fibroblasts, and human dermal fibroblasts (hDermFib). SEQ ID NO: 1 is depicted as Pep 1 and SEQ ID NO: 2 is depicted as Pep 2 in the Figure.

FIG. 16 is a schematic diagram depicting one method for covalently attaching a binding peptide to a PEG-linker group and then to a substrate comprising amino functional groups.

FIG. 17 is a graph showing the relative binding affinity of synthetic BMP binding peptides SEQ ID NOs: 54-56 for BMP2 (SEQ ID NO: 54 (Peptide 1); SEQ ID NO: 55 (Peptide 2); SEQ ID NO: 56 (Peptide 3)).

FIG. 18 is a bar graph showing the relative binding of BMP binding peptide SEQ ID NO: 55 to various members of the BMP family. BMP binding peptide SEQ ID NO: 55 (“Peptide” in the Figure) was analyzed along with a “No Peptide” control on BMP family members: BMP2, BMP3, BMP5, BMP6, GDF5, GDF7, TGFb1, and TGFb3, as well as PDGF-BB, laminin, and collagen.

FIG. 19 is a table showing the effect of single amino acid scanning mutagenesis of SEQ ID NO: 55 on BMP2 Binding Activity. The amino acid sequence of SEQ ID NO: 55 is shown accross the top of FIG. 19 and the amino acid substitutions at each position are shown on both the far right and the far left of the Figure for convenience. The symbols used in the table are as follows: (−) no binding; (+) moderate binding; (++) strong binding; (++) with stippled cells denotes strong, but non-specific binding; (++) with shaded cells denotes that only strong binders were found for that position; (nd) the amino acid substitution was not found in the phage tested.

### DETAILED DESCRIPTION

The methods and compositions of the presently disclosed subject matter are described in greater detail herein below.

### DEFINITIONS

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims. Thus, for example, reference to “a cell binding peptide” or reference to “a 1 unit polyethylene glycol (“mini-PEG″ or “MP”)” includes a plurality of such cell binding peptides or such polyethylene glycol units, and so forth.

The term “adipose tissue” as used herein, for the purposes of the specification and claims, includes the term “liposuction aspirate”. Therefore, the term “stromal vascular fraction of adipose tissue” also means “stromal vascular fraction of liposuction aspirate”.

The cell binding peptides and the BMP binding peptides of the presently disclosed subject matter are herein collectively referred to as the “binding peptides”. The term “cell binding peptide” is used herein, for the purposes of the specification and claims, to refer to an amino acid chain comprising a peptide that can bind to a cell and is set forth in any one of SEQ ID NOs: 1-53. In one embodiment, the presently disclosed subject matter provides a cell binding polypeptide, wherein the polypeptide comprises a cell binding peptide selected from the group consisting of SEQ ID NOs: 1-53, and wherein the polypeptide comprises from up to as many as 15, 16, 17, 18, 19, 20, 25, 30, 35, 40, 45, 50, 55, 60, 65, 70, or 75 amino acids, or any number of amino acids between 15 and 75 amino acids even if not specifically called out here. The cell binding peptides of the presently disclosed subject matter bind one or more of stem cells or fibroblasts. In addition, the term “stem cell binding peptide” is in some cases herein used interchangeably, for the purposes of the specification and claims, with the terms “cell binding peptide” and “adipose-derived stem cell (ASC) binding peptide” and “fibroblast binding peptide” as certain of the stem cell binding peptides also bind to fibroblasts.

The term “BMP binding peptide” is used herein, for the purposes of the specification and claims, to refer to an amino acid chain comprising a peptide that can bind to a bone morphogenic protein (BMP) and is set forth in any one of SEQ ID NOs: 54-184, 189-192, or 198-203. In one embodiment, the presently disclosed subject matter provides a BMP binding polypeptide, wherein the polypeptide comprises a BMP binding peptide selected from the group consisting of SEQ ID NOs: 54-184, 189-192, and 198-203, and wherein the polypeptide comprises from up to as many as 10, 11, 12, 13, 14, 15, 20, 25, 30, 35, 40, 45, 50, 55, 60, 65, 70, or 75 amino acids, or any number of amino acids between 10 and 75 amino acids even if not specifically called out here. The BMPs are members of the transforming growth factor beta (TGF-β) superfamily that share a set of conserved cysteine residues and a high level of sequence identity overall. The BMP's, including BMP-2 and BMP-7, have shown clinical benefit in the treatment of bone fractures and spine fusions. Preferably, the BMP binding peptides of the presently disclosed subject matter bind to one or more of BMP-2, BMP-4, BMP-6, or BMP-7.

In one embodiment, the implantable device for tissue repair comprises a biopolymer having an attached binding peptide. The term “biopolymer” is used herein, for the purposes of the specification and claims, to refer to a biopolymer suitable for use in the compositions and methods of the presently disclosed subject matter. In one embodiment, a binding peptide is covalently attached to the biopolymer. Biopolymers of the of the presently disclosed subject matter include, by non-limiting example, a collagen, an injectable collagen, a fibrillar collagen, a Type I collagen, a bovine collagen, a recombinant collagen, an animal-derived collagen, a gelatin, an elastin, a keratin, a silk, a polysaccharide, an agarose, a dextran, a cellulose derivative, an oxidized cellulose, an oxidized regenerated cellulose, a carboxymethylcellulose, a hydroxypropylmethylcellulose, a chitosan, a chitin, a hyaluronic acid, and derivatives and combinations thereof.

In one embodiment, implantable device can comprise any material and can be present in any form that is desirable and conducive to capturing cells onto the substrate such that the cells retain their native activity such as, for example, stem cells retaining their ability to differentiate into one or more cells of mesenchymal tissue lineage. Similarly, the implantable device can comprise any material and can be present in any form that is desirable and conducive to capturing BMPs onto the substrate such that the BMPs retain their biological BMP activity. The term “implantable device” generally refers to a structure that is introduced into a human or animal body to restore a function of a damaged tissue or to provide a new function. Representative implantable devices for soft tissue repair include, but are not limited to, a gel, a hydrogel, an injectable material, an extracellular matrix, a decellularized tissue, a dermal matrix, a small intestinal submucosa (SIS), an acellular human dermis, an acellular porcine dermis, an acellular bovine dermis, an acellular myocardium, a cardiac patch, a heart valve, a surgical mesh, a skin graft, an injectable for dermal tissue augmentation, a dural graft, a graft for foot ulcer repair, a hernia repair graft, a graft for abdominal repair, a tendon wrap, a tendon augmentation graft, a graft for rotator cuff repair, a graft or mesh for breast reconstruction, a graft or mesh for pelvic floor reconstruction, a graft for medial collateral ligament repair, a graft for anterior cruciate ligament repair, a composite surgical mesh comprising a synthetic biopolymer and a biopolymer, and derivatives and combinations thereof. In one embodiment, the implantable device for soft tissue repair is in the form of an injectable or a formed piece. In general, the shape and size of the implantable device will preferably closely mimic the size and shape of the defect it is trying to repair. In one embodiment, the implantable device will be in the shape of a formed piece. For a rotator cuff repair, for example, it may be preferable to use a formed piece in a sheet configuration such as a rectangular patch, or a circular patch that can be cut to size. In one embodiment, the implantable device is in an injectable form in which it will have a viscosity low enough to allow it to be injected into a defect site using a large bore syringe or a syringe/needle combination.

In one embodiment, the tissue for repair is bone tissue and the implantable device for bone tissue repair includes implantable devices comprising the biopolymer that are a bone graft material further comprising a ceramic. The terms “implantable device”, “bone graft material”, “bone void filler”, and “bone graft substitute” are herein used interchangeably for the purposes of the specification and claims to refer to an implantable medical device for promoting bone formation. In one embodiment, the bone graft material comprises a ceramic and a polymer, wherein the polymer comprises a covalently attached BMP binding peptide. In one embodiment, the bone graft material of the presently disclosed subject matter is a composite of a ceramic (e.g., TCP) and a biopolymer and, therefore, the terms “implantable device”, “bone graft material”, “bone void filler”, “bone graft substitute”, “composite”, and “collagen/TCP composite” are also in some cases used interchangeably for the purposes of the specification and claims. The term “ceramic” is used herein, for the purposes of the specification and claims, to refer to particulate ceramic mineral or inorganic filler useful for promoting bone formation. The ceramics of the presently disclosed subject matter include, by non-limiting example, synthetic and naturally occurring inorganic fillers such as calcium phosphate, calcium phosphate cement, biocompatible magnesium doped calcium phosphates, calcium carbonate, calcium sulfate, barium carbonate, barium sulfate, alphatricalcium phosphate (α-TCP), tricalcium phosphate (TCP), betatricalcium phosphate (β-TCP), hydroxyapatite (HA), biphasic calcium phosphate, biphasic composite between HA and β-TCP, alumina, zirconia, bioglass, biocompatible silicate glasses, biocompatible phosphate glasses, bone particles, and combinations and mixtures thereof. In certain embodiments the ceramic comprises a polymorph of calcium phosphate. Preferably, the ceramic is beta-tricalcium phosphate.

In one embodiment, the bone graft material comprises a composite of a ceramic and a biopolymer. In one embodiment, the ceramic and the biopolymer are present at a weight ratio ranging from about 10:1 ceramic to biopolymer to about 2:1 ceramic to biopolymer. In one embodiment, the weight ratio of the ceramic to the biopolymer is from about 2:1 (about 66% ceramic to about 33% biopolymer), from about 3:1 (about 75% ceramic to about 25% biopolymer), from about 4:1 (about 80% ceramic to about 20% biopolymer), from about 9:1 (about 90% ceramic to about 10% biopolymer), from about 10:1 (about 99% ceramic to about 1% biopolymer).

The implantable devices for tissue repair of the presently disclosed subject matter comprise a biopolymer having an attached binding peptide. A number of acellular extracellular matrices and composites of absorbable and non-absorbable materials for soft tissue repair that comprise one or more of the biopolymers listed herein above are discussed in Grevious et al., Clin Plastic Surg, 2006, 33:181-97; Butler, C. E., Clin Plastic Surg, 2006, 33:199-211; Badylak, S. F., Biomaterials, 2007, 28:3587-93; Longo et al., British Medical Bulletin, 2010, 94:165-88; Gentleman et al., Biomaterials, 2003, 24:3805-13; and U.S. Pat. No. 6,063,120; each of which is herein incorporated by reference in its entirety. The extracellular matrices and composites described in the foregoing articles that comprise one or more of the biopolymers listed herein above are implantable devices to which a binding peptide of the presently disclosed subject matter is covalently attached.

The term “substrate” is used, for the purposes of the specification and claims, to refer to any material that is biologically compatible with cells and/or growth factors and to which a binding peptide can be attached for the purpose of capturing target cells and/or growth factors onto the substrate. Representative substrates comprise one or more of metal, glass, plastic, synthetic matrix, silica gel, polymer, biopolymer, or derivatives or combinations thereof. The term “attached” in reference to a binding peptide of the presently disclosed subject matter being “attached” to a substrate and/or a biopolymer means, for the purposes of the specification and claims, a binding peptide being immobilized on the substrate and/or biopolymer by means that will enable capture of the binding peptide target (i.e. cell or BMP) onto the substrate and/or biopolymer. The binding peptide attached to the substrate can be one or more of a cell binding peptide or a growth factor binding peptide, or combinations thereof. In one embodiment the substrate is in the form of an implantable device. Therefore, the terms “substrate” and “implantable device” are herein used interchangeably, for the purposes of the specification and claims. In one embodiment, the implantable device for tissue repair comprises a biopolymer having an attached binding peptide. Accordingly, the term “substrate” is in some cases herein used interchangeably with the term “biopolymer”. For example, in certain embodiments depicted in the drawings when referring to the attachment of a binding peptide to a “substrate” it is meant that attachment of the binding peptide is to a biopolymer comprised in the substrate.

In the case of the cell binding peptides, the cell binding peptides can be “attached” to the substrate or biopolymer by means that will enable capture of cells onto the biopolymer such that the stem cells retain their native activity. In the case of the BMP binding peptides, the BMP binding peptides can be “attached” to the biopolymer by any means that will enable capture of BMPs onto the implantable device such that the BMPs retain their biological BMP activity. The BMP binding peptides are covalently attached to the biopolymer. The term “attached” in reference to a BMP binding peptide of the presently disclosed subject matter being attached to a biopolymer means, for the purposes of the specification and claims, a binding peptide being immobilized on the biopolymer by covalent attachment by any means that will enable binding of BMP onto the peptide-modified biopolymer such that the bound BMP retains biological growth factor activity. A binding peptide can be attached to a biopolymer by any one of covalent bonding, non-covalent bonding including, one or more of hydrophobic interactions, Van der Weals forces, hydrogen bonds, ionic bonds, magnetic force, or avidin-, streptavidin-, and Neutravidin-biotin bonding.

The binding peptides of the presently disclosed subject matter can include naturally occurring amino acids, synthetic amino acids, genetically encoded amino acids, non-genetically encoded amino acids, and combinations thereof; however, an antibody is specifically excluded from the scope and definition of a binding peptide of the presently disclosed subject matter. A binding peptide used in accordance with the presently disclosed subject matter can be produced by chemical synthesis, recombinant expression, biochemical or enzymatic fragmentation of a larger molecule, chemical cleavage of larger molecule, a combination of the foregoing or, in general, made by any other method in the art, and preferably isolated.

Binding peptides useful in the presently disclosed subject matter also include peptides having one or more substitutions, additions, and/or deletions of residues relative to the sequence of an exemplary cell binding peptide or BMP binding peptide shown herein at Tables 1-5, as long as the binding properties of the exemplary binding peptides to their targets are substantially retained. Thus, the binding peptides include those that differ from the exemplary sequences by about 1, 2, 3, 4, 5, 6, 7, 8, 9, or 10 amino acids, and include binding peptides that share sequence identity with the exemplary peptide of at least 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, or greater sequence identity. Sequence identity can be calculated manually or it can be calculated using a computer implementation of a mathematical algorithm, for example, GAP, BESTFIT, BLAST, FASTA, and TFASTA, or other programs or methods known in the art. Alignments using these programs can be performed using the default parameters. A binding peptide can have an amino acid sequence consisting essentially of a sequence of an exemplary binding peptide or a binding peptide can have one or more different amino acid residues as a result of substituting an amino acid residue in the sequence of the exemplary binding peptide with a functionally similar amino acid residue (a “conservative substitution”); provided that the peptide containing the conservative substitution will substantially retain the binding activity of the exemplary binding peptide not containing the conservative substitution. Examples of conservative substitutions include the substitution of one non-polar (hydrophobic) residue such as alanine, isoleucine, valine, leucine, or methionine for another; the substitution between asparagine and glutamine, the substitution of one large aromatic residue such as tryptophan, tyrosine, or phenylalanine for another; the substitution of one small polar (hydrophilic) residue for another such as between glycine, threonine, serine, and proline; the substitution of one basic residue such as lysine, arginine, or histidine for another; or the substitution of one acidic residue such as aspartic acid or glutamic acid for another.

Accordingly, binding peptides useful in the presently disclosed subject matter include those peptides that are conservatively substituted variants of the binding peptides set forth in SEQ ID NOs: 1-49 (cell binding peptides) and SEQ ID NOs: 54-184 and 189-192 (BMP binding peptides), and those peptides that are variants having at least 65% sequence identity or greater to the binding peptides set forth in SEQ ID NOs: 1-49 and SEQ ID NOs: 54-184 and 189-192, wherein all of the variant binding peptides useful in the presently disclosed subject matter substantially retain the ability to bind to their target.

Binding peptides can include L-form amino acids, D-form amino acids, or a combination thereof. Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; ornithine; and 3-(3,4-dihydroxyphenyl)-L-alanine (“DOPA”). Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

Further, a binding peptide according to the presently disclosed subject matter can include one or more modifications, such as by addition of chemical moieties, or substitutions, insertions, and deletions of amino acids, where such modifications provide for certain advantages in its use, such as to facilitate attachment to the biopolymer with or without a spacer or to improve peptide stability. The term “spacer” is used herein, for the purposes of the specification and claims, to refer to a compound or a chemical moiety that is optionally inserted between a binding peptide and the biopolymer. In some embodiments, the spacer also serves the function of a linker (i.e. to attach the binding peptide to the biopolymer). Therefore, the terms “linker” and “spacer” can be used interchangeably herein, for the purposes of the specification and claims, when performing the dual functions of linking (attaching) the peptide to the biopolymer and spacing the binding peptide from the biopolymer. In some cases the spacer can serve to position the binding peptide at a distance and in a spatial position suitable for binding and capture and/or in some cases the spacer can serve to increase the solubility of the binding peptide. Spacers can increase flexibility and accessibility of the binding peptide to its target, as well as increase the binding peptide density on the biopolymer surface. Virtually all chemical compounds, moieties, or groups suitable for such a function can be used as a spacer unless adversely affecting the binding behavior to such an extent that binding of the target to the binding peptides is prevented or substantially impaired. Thus, the term “binding peptide” encompasses any of a variety of forms of binding peptide derivatives including, for example, amides, conjugates with proteins, conjugates with polyethylene glycol or other biopolymers, cyclic peptides, biopolymerized peptides, peptides having one or more amino acid side chain group protected with a protecting group, and peptides having a lysine side chain group protected with a protecting group. Any binding peptide derivative that has substantially retained target binding characteristics can be used in the practice of the presently disclosed subject matter.

Further, a chemical group can be added to the N-terminal amino acid of a binding peptide to block chemical reactivity of the amino terminus of the peptide. Such N-terminal groups for protecting the amino terminus of a peptide are well known in the art, and include, but are not limited to, lower alkanoyl groups, acyl groups, sulfonyl groups, and carbamate forming groups. Preferred N-terminal groups can include acetyl, 9-fluorenylmethoxycarbonyl (Fmoc), and t-butoxy carbonyl (Boc). A chemical group can be added to the C-terminal amino acid of a synthetic binding peptide to block chemical reactivity of the carboxy terminus of the peptide. Such C-terminal groups for protecting the carboxy terminus of a peptide are well known in the art, and include, but are not limited to, an ester or amide group. Terminal modifications of a peptide are often useful to reduce susceptibility by protease digestion, and to therefore prolong a half-life of a binding peptide in the presence of biological fluids where proteases can be present. In addition, as used herein, the term “binding peptide” also encompasses a peptide wherein one or more of the peptide bonds are replaced by pseudopeptide bonds including but not limited to a carba bond (CH₂—CH₂), a depsi bond (CO—O), a hydroxyethylene bond (CHOH—CH₂), a ketomethylene bond (CO—CH₂), a methylene-oxy bond (CH₂—O), a reduced bond (CH₂—NH), a thiomethylene bond (CH₂—S), an N-modified bond (—NRCO), and a thiopeptide bond (CS—NH).

In one embodiment, the binding peptides are covalently attached to the substrate and/or biopolymer comprised in the implantable device. For ease of reading this discussion of covalent attachment, the term “substrate” will be hereby be used to represent the phrase “substrate and/or biopolymer”. In one embodiment, the linkers/spacers for use in attaching binding peptides to the substrate have at least two chemically active groups (functional groups), of which one group binds to the substrate, and a second functional group binds to the binding peptide or in some cases it binds to the “spacer” already attached to the binding peptide. Preferably, the attachment of the binding peptides to the substrate is effected through a spacer. Virtually all chemical compounds, moieties, or groups suitable for such a function can be used as a spacer unless adversely affecting the peptide binding behavior to such an extent that binding of the target to the binding peptides is prevented or substantially impaired.

Again, the terms “linker” and “spacer” can be used interchangeably herein, for the purposes of the specification and claims, when performing the dual functions of linking (attaching) the binding peptide to the substrate and spacing the peptide from the substrate. In many embodiments herein, the linkers used to attach the binding peptide to the substrate function as both a linker and a spacer. For example, a linker molecule can have a linking functional group on either end while the central portion of the molecule functions as a spacer. The binding peptides of the presently disclosed subject matter can comprise a functional group that is intrinsic to the binding peptide (e.g., amino groups on lysine), or the functional group can be introduced into the binding peptide by chemical modification to facilitate covalent attachment of the binding peptide to the substrate. Similarly, the substrate can comprise a functional group that is intrinsic to the biopolymer (e.g., amino groups on collagen), or the biopolymer can be modified with a functional group to facilitate covalent attachment to the binding peptide. The binding peptide can be covalently attached to the substrate with or without one or more spacer molecules.

For example, linkers/spacers are known to those skilled in the art to include, but are not limited to, chemical compounds (e.g., chemical chains, compounds, reagents, and the like). The linkers/spacers may include, but are not limited to, homobifunctional linkers/spacers and heterobifunctional linkers/spacers. Heterobifunctional linkers/spacers, well known to those skilled in the art, contain one end having a first reactive functionality (or chemical moiety) to specifically link a first molecule (e.g, substrate), and an opposite end having a second reactive functionality to specifically link to a second molecule (e.g, binding peptide). It is evident to those skilled in the art that a variety of bifunctional or polyfunctional reagents, both homo- and hetero-functional can be employed as a linker/spacer with respect to the presently disclosed subject matter such as, for example, those described in the catalog of the PIERCE CHEMICAL CO., Rockford, Ill.; amino acid linkers/spacers that are typically a short peptide of between 3 and 15 amino acids and often containing amino acids such as glycine, and/or serine; and wide variety of biopolymers including, for example, polyethylene glycol. In one embodiment, representative linkers/spacers comprise multiple reactive sites (e.g., polylysines, polyornithines, polycysteines, polyglutamic acid and polyaspartic acid) or comprise substantially inert peptide spacers (e.g., polyglycine, polyserine, polyproline, polyalanine, and other oligopeptides comprising alanyl, serinyl, prolinyl, or glycinyl amino acid residues). In one embodiment, representative spacers between the reactive end groups in the linkers include, by non-limiting example, the following functional groups: aliphatic, alkene, alkyne, ether, thioether, amine, amide, ester, disulfide, sulfone, and carbamate, and combinations thereof. The length of the spacer can range from about 1 atom to 200 atoms or more. In one embodiment, linkers/spacers comprise a combination of one or more amino acids and another type of spacer or linker such as, for example, a biopolymeric spacer.

Suitable biopolymeric spacers/linkers are known in the art, and can comprise a synthetic biopolymer or a natural biopolymer. Representative synthetic biopolymer linkers/spacers include but are not limited to polyethers (e.g., poly(ethylene glycol) (“PEG”), 11 unit polyethylene glycol (“PEG10”), or 1 unit polyethylene glycol (“mini-PEG” or “MP”), poly(propylene glycol), poly(butylene glycol), polyesters (e.g., polylactic acid (PLA) and polyglycolic acid (PGA)), polyamines, polyamides (e.g., nylon), polyurethanes, polymethacrylates (e.g., polymethylmethacrylate; PMMA), polyacrylic acids, polystyrenes, and polyhexanoic acid, and combinations thereof. Biopolymeric spacers/linkers can comprise a diblock biopolymer, a multi-block cobiopolymer, a comb biopolymer, a star biopolymer, a dendritic or branched biopolymer, a hybrid linear-dendritic biopolymer, a branched chain comprised of lysine, or a random cobiopolymer. A spacer/linker can also comprise a mercapto(amido)carboxylic acid, an acrylamidocarboxylic acid, an acrlyamido amidotriethylene glycolic acid, 7-aminobenzoic acid, and derivatives thereof.

In one embodiment, the binding peptide comprises one or more modifications to the peptide N-terminus, peptide C-terminus, or within the peptide amino acid sequence, to facilitate covalent attachment of the binding peptide to a substrate device with or without a spacer. The binding peptides can comprise one or more modifications including, but not limited to, addition of one or more groups such as hydroxyl, thiol, carbonyl, carboxyl, ester, carbamate, hydrazide, hydrazine, isocyanate, isothiocyanate, amino, alkene, dienes, maleimide, α,β-unsaturated carbonyl, alkyl halide, azide, epoxide, N-hydroxysuccinimide (NHS) ester, lysine, or cysteine. In addition, a binding peptide can comprise one or more amino acids that have been modified to contain one or more chemical groups (e.g., reactive functionalities such as fluorine, bromine, or iodine) to facilitate linking the binding peptide to a spacer molecule or to the substrate to which the binding peptide will be attached.

The binding peptides can be covalently attached to the substrate through one or more anchoring (or linking) groups on the substrate and the binding peptide. The binding peptides of the presently disclosed subject matter can comprise a functional group that is intrinsic to the binding peptide, or the binding peptide can be modified with a functional group to facilitate covalent attachment to the substrate with or without a spacer. Representative anchoring (or linking) groups include by non-limiting example hydroxyl, thiol, carbonyl, carboxyl, ester, carbamate, hydrazide, hydrazine, isocyanate, isothiocyanate, amino, alkene, dienes, maleimide, α,β-unsaturated carbonyl, alkyl halide, azide, epoxide, NHS ester, lysine, and cysteine groups on the surface of the substrate. The anchoring (or linking) groups can be intrinsic to the material of the substrate (e.g., amino groups on a collagen or on a polyamine-containing biopolymer) or the anchoring groups can be introduced into the substrate by chemical modification.

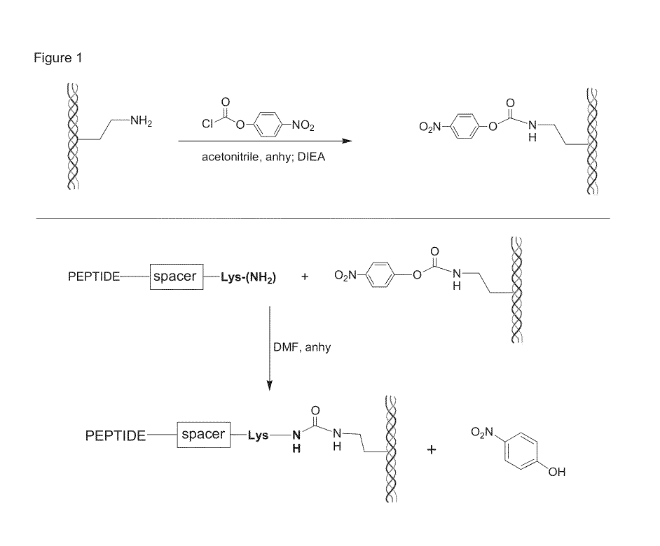

By way of non-limiting example, in one embodiment, a binding peptide is attached to a substrate in a two step process (see FIG. 1; Mikulec & Puleo, 1996, J. Biomed. Mat. Res., Vol 32, 203-08). In the first step, the anchoring (or linking) groups (i.e., amino groups on a collagen for example) on the surface of a substrate are activated by an acylating reagent (4-nitrophenyl chloroformate). In the second step, a lysine residue which has been introduced along with a PEG10 spacer at the C-terminus of a binding peptide is reacted with the activated chloroformate intermediate on the substrate surface, resulting in attachment of the binding peptide to the substrate.

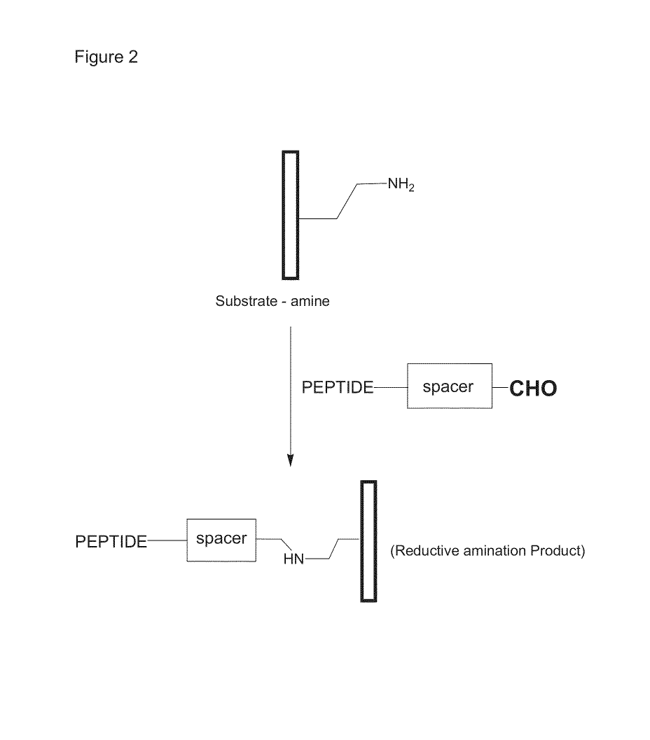

By way of non-limiting example, in one embodiment, a binding peptide is covalently attached to a substrate comprising an amino functional group (see FIG. 2). FIG. 2 exemplifies attachment of a binding peptide comprising an aldehyde group at one terminus to a substrate that comprises an amino functional group. The binding peptide comprising an aldehyde functional group is treated with the substrate amino groups under reductive amination conditions to give attached binding peptide. In another embodiment not depicted in FIG. 2, a binding peptide comprising an amine functional group is reacted with the substrate amino groups via a homobifunctional linker such as, for example, glutaraldehyde, to yield a covalently attached binding peptide (Simionescu et. al., 1991, J. Biomed Mater. Res., 25:1495-505).

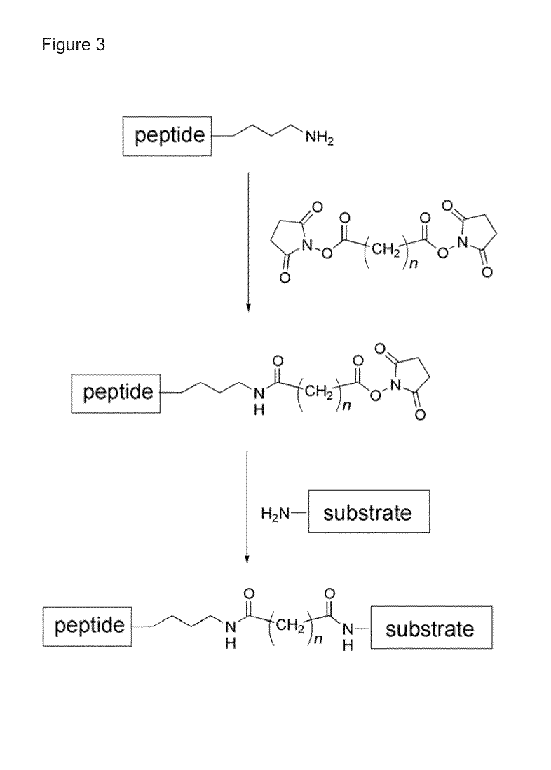

By way of non-limiting example, in one embodiment, a homobifunctional linker possessing N-hydroxysuccinimide esters at both ends is reacted at one end with the binding peptide having an amino group (FIG. 3). The binding peptide with attached linking group is then reacted through the remaining N-hydroxysuccinimide ester with an amino group on the substrate to form a peptide-substrate conjugate (FIG. 3). The homobifunctional N-hydroxysuccinimide ester depicted in FIG. 3 is BS³ crosslinking reagent (THERMO SCIENTIFIC, Rockford, Ill.). As stated herein previously, the length and type of spacer groups between the two reactive end groups on the NHS ester can vary.

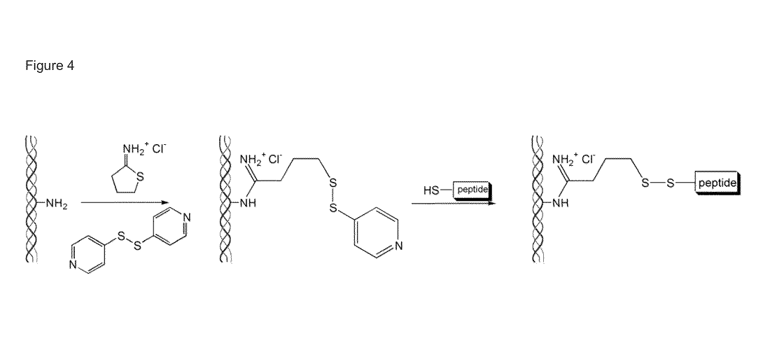

By way of non-limiting example, in one embodiment, a binding peptide is covalently attached to a substrate having amino functional groups in a two-step process using a disulfide linkage (see FIG. 4; Hermanson, G. T. Bioconjugate Techniques; Academic Press: San Diego, 1996; pp. 150-151). First, the substrate containing amino groups is reacted with 2-iminothiolane resulting in the introduction of thiol groups on the substrate. Simultaneous addition of 4,4′-dithiodipyridine or 6,6′-dithiodinicotinic acid results in rapid capping of the newly-introduced thiol as a pyridyl disulfide. Second, the binding peptide containing a free thiol is attached covalently to the substrate through a thiol-disulfide exchange resulting in a disulfide bond between the substrate and binding peptide.

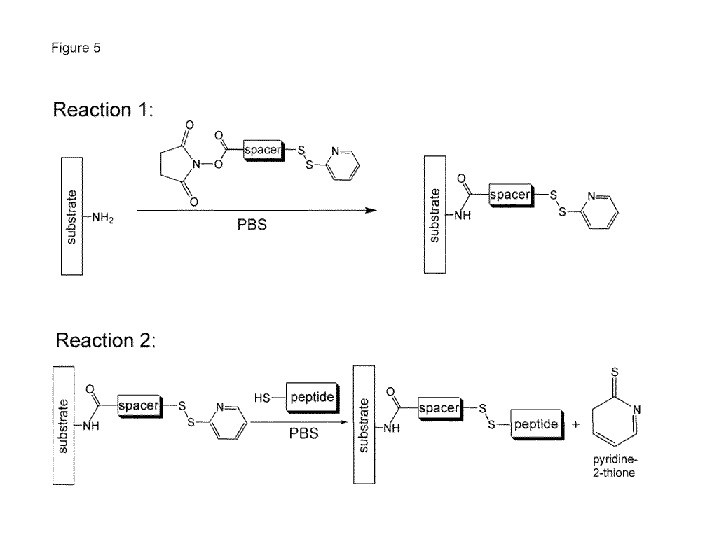

By way of non-limiting example, in one embodiment, a binding peptide is attached covalently to a substrate comprising amino functional groups in a similar process using a disulfide linkage (see FIG. 5; Carlsson et al., 1978, Biochem. J., 173:723-37). The substrate is first functionalized with amine groups using known methods (if the amino groups are not intrinsic to the material of the substrate). Next, a thiol-cleavable, heterobifunctional (amine- and sulfhydryl-reactive) compound (LC-SPDP; THERMO SCIENTIFIC, Rockford, Ill.) is reacted with the amino-functionalized substrate. The binding peptide is reacted with the LC-SPDP modified substrate.

By way of non-limiting example, in one embodiment, a binding peptide is attached covalently to a substrate via a thioether bond formed by reaction of a thiol and maleimide (O'Sullivan et al., 1979, Anal. Biochem., 100:100-8). In one embodiment, the maleimide is added to a substrate comprising amino functional groups and then the modified substrate is reacted with a binding peptide having a free thiol group. Alternatively, in one embodiment, the same chemical scheme is utilized but with the substrate modified with a thiol group and the binding peptide modified with the maleimido group.

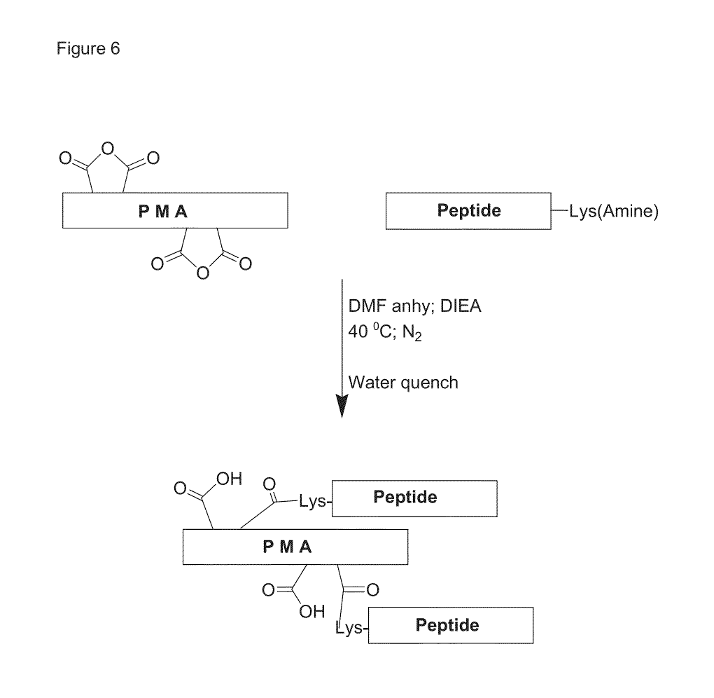

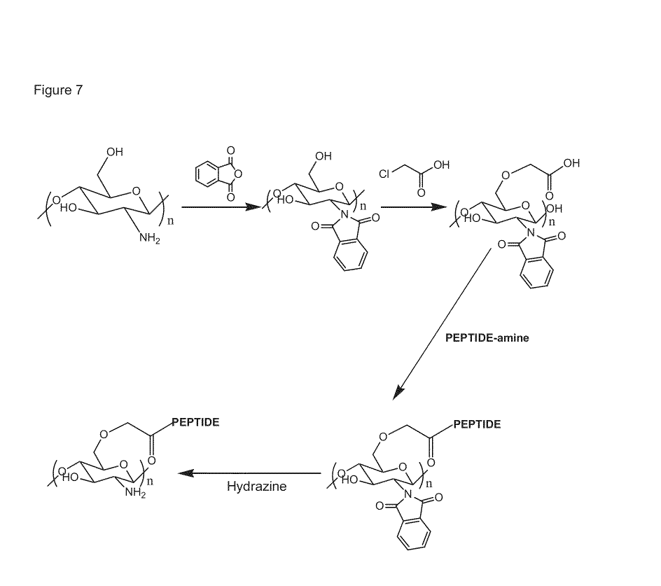

By way of non-limiting example, in one embodiment, a binding peptide is covalently attached through a non-backbone anhydride group of a polyanhydride biopolymer, polymaleic acid (PMA), through a reactive lysine group on the binding peptide shown in the schematic diagram in FIG. 6 (Pompe, et al., 2003, Biomacromolecules, 4(4):1072-9).

By way of non-limiting example, in one embodiment, a binding peptide is covalently attached to a chitosan. The chemical scheme is shown in FIG. 7. First, the amino group on chitosan is protected with phthaloyl group. The hydroxyl group on chitosan is then reacted with chloroacetic acid to give an acid handle on chitosan. The binding peptide amine is coupled to the acid group on the chitosan to give the binding peptide-chitosan conjugate. The phthaloyl group is then removed using hydrazine.

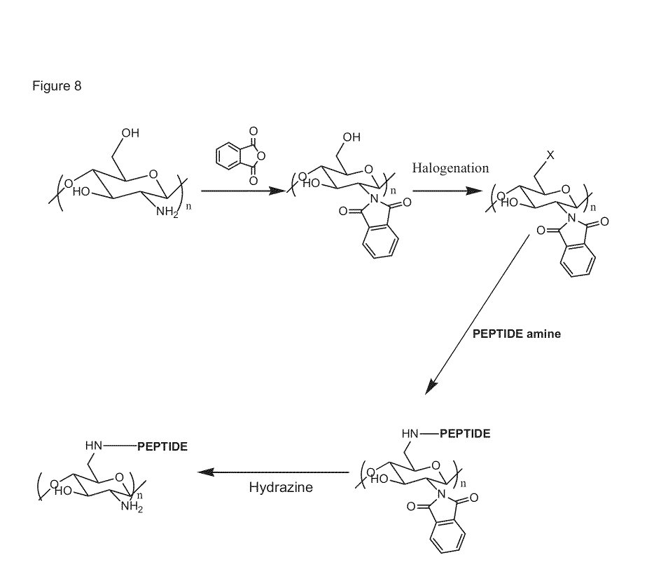

By way of non-limiting example, in one embodiment a binding peptide is covalently attached to a chitosan. The chemical scheme is shown in FIG. 8. First, the amino group on chitosan is protected with a phthaloyl group. The hydroxyl group on chitosan is then converted to a bromo group under standard halogenation conditions. The binding peptide amine is reacted with halogenated chitosan to give the binding peptide-chitosan conjugate. The phthaloyl group is finally removed by reacting with hydrazine.

By way of non-limiting example, in one embodiment a binding peptide is covalently attached to chitosan through the amino group on chitosan. For example, a chemical scheme using a homobifunctional N-hydroxysuccinimide ester, such as that described for FIG. 3, is useful for attaching the binding peptide through the amino group on chitosan.

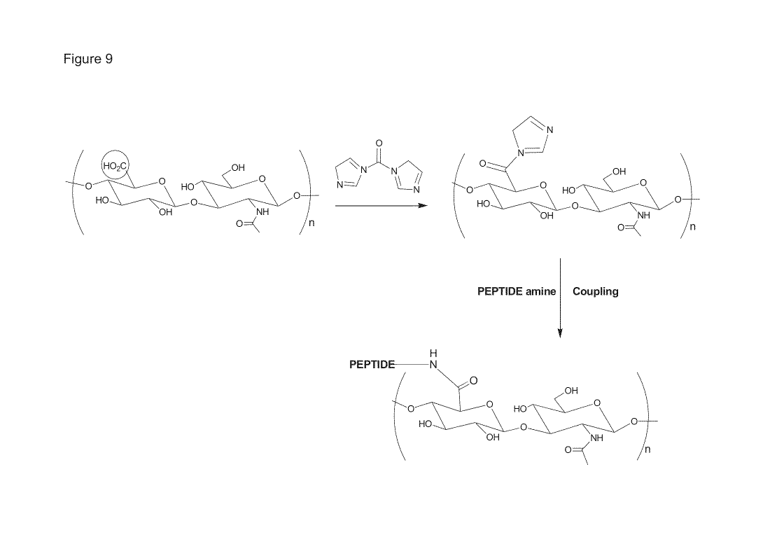

By way of non-limiting example, in one embodiment a binding peptide is covalently attached to a hyaluronan (HA). The chemical scheme is shown in FIG. 9. The hyaluronan is chemically modified at the carboxylic acid group on the glucuronate units. The carboxylic group is activated using carbonyl diimidazole (CD). The activated HA is then reacted with the amino group of binding peptide to yield the peptide-HA conjugate.

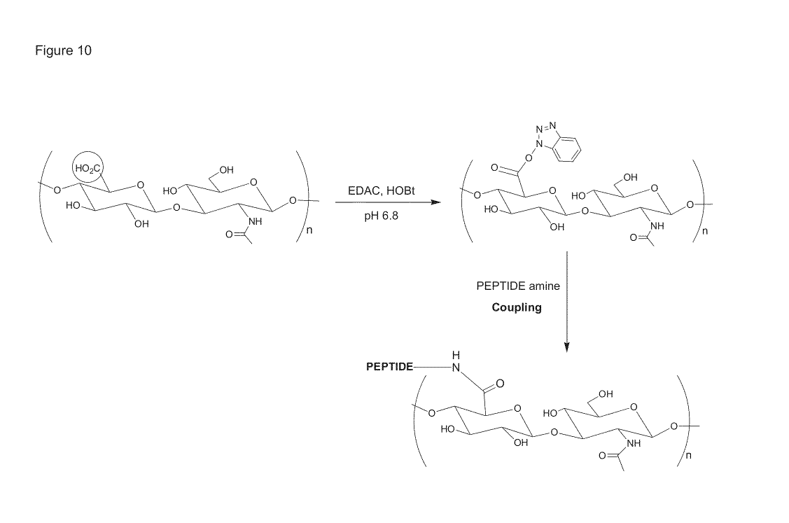

By way of non-limiting example, in one embodiment, a binding peptide is covalently attached to a hyaluronan (HA). The chemical scheme is shown in FIG. 10. Hyaluronan is chemically modified at the carboxylic acid group on the glucuronate units. The carboxylic group is activated using water soluble carbodiimide such as 1-ethyl-3-(3-dimethylaminopropyl) carbodimide (EDC) along with HOBt. The activated HA is coupled with the amino group of a binding peptide to yield the peptide-HA conjugate.

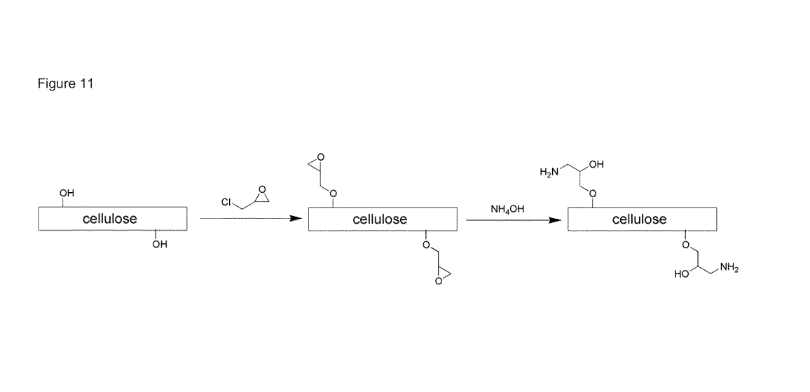

By way of non-limiting example, in one embodiment, a binding peptide is covalently attached to cellulose. The chemical scheme is shown in FIG. 11. Hydroxyl groups on the polysaccharide are first reacted with epichlorohydrin to introduce an epoxide. Ring opening of the epoxide by reaction with aqueous ammonia provides free amino groups that can function as anchors for peptide conjugation using chemistry described in previous embodiments (Matsumoto, et al. (1980) J. Biochem., 87: 535-540).

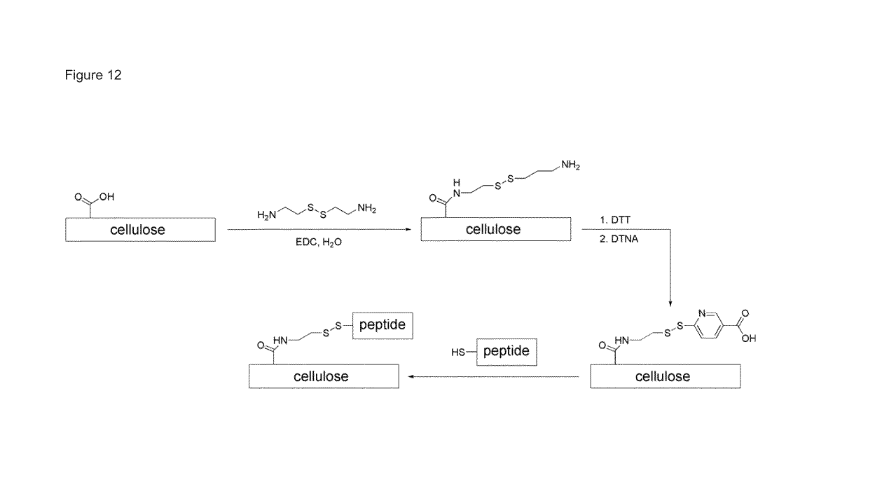

By way of non-limiting example, in one embodiment, a binding peptide is covalently attached to oxidized cellulose. The chemical scheme is shown in FIG. 12. Sulfhydryl groups are introduced by reaction of carboxylates on the oxidized cellulose with cystamine and EDC followed by reduction with dithiothreitol (DTT). Activation of sulfhydryls with 6,6′-dithiodinicotinic acid (DTNA) followed by a sulfhydryl-containing binding peptide results in covalent attachment of the peptide to the oxidized cellulose through a disulfide bond. In another embodiment not depicted in FIG. 12, the sulfhydryl modified oxidized cellulose is reacted with a maleimide or other Michael acceptor on the binding peptide resulting in covalent attachment through a thioether bond. In another embodiment not depicted in FIG. 12, carboxyl groups on oxidized cellulose are activated with EDC and 1-hydroxybenzotriazole (HOBt) followed by reaction with cell binding peptide containing a free amine group. This results in conjugation of peptide to the oxidized cellulose through an amide bond (this chemistry is exemplified in FIG. 10). In another embodiment not depicted in FIG. 12, a cell binding peptide can be covalently attached to oxidized cellulose through the aldehyde groups on the oxidized cellulose. In this example, a cell binding peptide having a free amine undergoes reductive amination with the aldehyde group on the biopolymer substrate to yield an amine bond as shown in FIG. 2 (the chemistry is the same as that in FIG. 2 except that the functional groups on the biopolymer substrate and cell binding peptide are reversed).

By way of non-limiting example, in one embodiment, a cell binding peptide can be covalently attached to an oxidized dextran biopolymer substrate by reductive amination as described above for oxidized cellulose. More specifically, a cell binding peptide having a free amine undergoes reductive amination with the aldehyde group on the biopolymer substrate to yield an amine bond as shown in FIG. 2 (the chemistry is the same as that in FIG. 2 except that the functional groups on the biopolymer substrate and cell binding peptide are reversed).

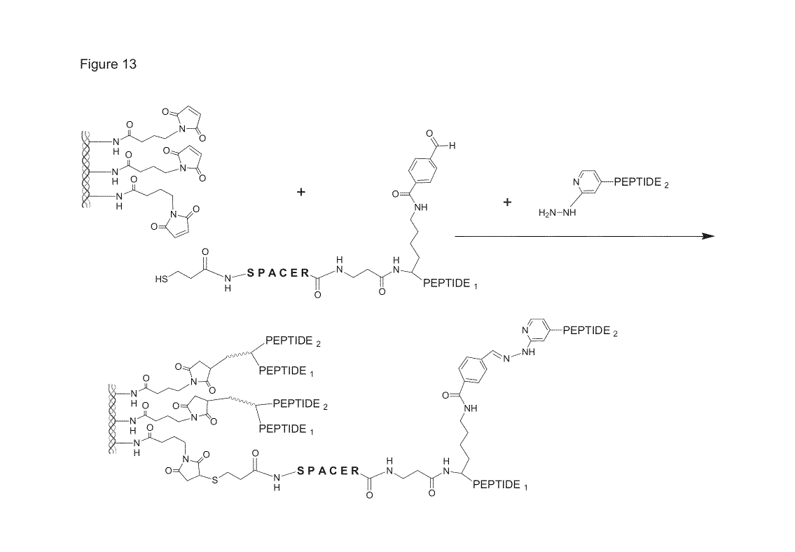

By way of non-limiting example, in one embodiment, more than one binding peptide is attached to a substrate. Attaching multiple binding peptides to a single substrate is only limited by practical considerations related to the method of attachment. For example, in one embodiment, two different binding peptides are covalently attached to a substrate using any of the chemical schemes shown in FIGS. 1-12. In each of the chemical schemes depicted in FIGS. 1-12, the substrate having a functional group is reacted with two or more different binding peptides that each comprise a functional group to covalently attach the two or more binding peptides to the substrate based on simple competition between the binding peptides. In particular, for example, in the case of the chemical schemes depicted in FIGS. 1 and 2, the modified substrate is reacted with two or more different binding peptides that each comprise an amino group or an aldehyde group (i.e., the two different binding peptides replace the single peptide depicted in FIGS. 1 and 2), to covalently attach the two or more binding peptides to the substrate through the amino or aldehyde group, respectively. In the case of the chemical schemes depicted in FIGS. 4 and 5, the modified substrate is reacted with two or more different binding peptides that each comprise a thiol group, to covalently attach the two or more binding peptides to the substrate through the thiol group (i.e., the “HS-Peptide” in FIGS. 4 and 5 in this embodiment represents two or more different binding peptides). By way of non-limiting example, in one embodiment, two different binding peptides are covalently attached to a substrate comprising amino groups using the chemical scheme shown in FIG. 13. In this embodiment, the amino groups on the substrate are modified with maleimido groups. The modified substrate is then reacted with a binding peptide comprising both a thiol group and an aldehyde group to covalently attach the binding peptide to the substrate through the thiol group. Next, the substrate-binding peptide conjugate is reacted with another binding peptide having a hydrazine group, to give a second covalent bond through the aldehyde-hydrazine (see FIG. 13). Alternatively, in one embodiment, the same chemical scheme is utilized but with the substrate modified with a thiol group and the binding peptide modified with the maleimido group. In addition to using this scheme to covalently attach different binding peptides, the scheme is also useful for attaching the same binding peptide.

In one embodiment, the presently disclosed subject matter provides cell binding peptides. In one embodiment, the cell binding peptides comprise a sequence selected from the group consisting of SEQ ID NOs: 1-53. The cell binding peptides bind to one or more of fibroblasts or stem cells. In one embodiment, the cell binding peptides comprise a sequence selected from the group consisting of SEQ ID NOs: 1-49, conservatively substituted variants of SEQ ID NOs: 1-49, and variants having at least 70% sequence identity to SEQ ID NOs: 1-49, wherein the variant cell binding peptide substantially retains the ability to bind cells. In one embodiment, a cell binding polypeptide is provided, wherein the polypeptide comprises a cell binding peptide selected from the group consisting of SEQ ID NOs: 1-53, wherein the polypeptide comprises from up to as many as 15, 16, 17, 18, 19, 20, 25, 30, 35, 40, 45, 50, 55, 60, 65, 70, or 75 amino acids, or any number of amino acids between 15 and 75 amino acids even if not specifically enumerated here, wherein the cell binding polypeptide substantially retains the ability to bind cells.

In one embodiment, the presently disclosed subject matter provides BMP binding peptides. In one embodiment, the BMP binding peptides comprise a sequence selected from the group consisting of SEQ ID NOs: 54-184, 189-192 and 198-203. In one embodiment, the BMP binding peptides comprise a sequence selected from the group consisting of SEQ ID NOs: 54-184 and 189-192, conservatively substituted variants of SEQ ID NOs: 54-184 and 189-192, and variants having at least 90% sequence identity to SEQ ID NOs: 54-184 and 189-192, wherein the variant BMP binding peptide substantially retains the ability to bind BMP. In one embodiment, a BMP binding polypeptide is provided, wherein the polypeptide comprises a BMP binding peptide selected from the group consisting of SEQ ID NOs: 54-184, 189-192 and 198-203, wherein the polypeptide comprises from up to as many as 10, 11, 12, 13, 14, 15, 20, 25, 30, 35, 40, 45, 50, 55, 60, 65, 70, or 75 amino acids, or any number of amino acids between 10 and 75 amino acids even if not specifically enumerated here, wherein the BMP binding polypeptide substantially retains the ability to bind BMP.

In one embodiment, the binding peptides comprise one or more modifications to the peptide N-terminus, peptide C-terminus, or within the peptide amino acid sequence. In one embodiment the modification is selected from the group consisting of aldehyde group, hydroxyl group, thiol group, amino group, amino acids, lysine, cysteine, acetyl group, biopolymers, synthetic biopolymers, polyethers, poly(ethylene glycol) (“PEG”), an 11 unit polyethylene glycol (“PEG10”), and a 1 unit polyethylene glycol (“mini-PEG” or “MP”), and combinations thereof.

In one embodiment, an implantable device for tissue repair is provided comprising a biopolymer having a covalently attached cell binding peptide and/or BMP binding peptide, wherein the cell binding peptide comprises a sequence set forth in any one of SEQ ID NOs: 1-53, and wherein the BMP binding peptide comprises a sequence selected from the group consisting of SEQ ID NOs: 54-184, 189-192, and 198-203. In one embodiment, the binding peptide is attached to the biopolymer with or without a spacer. In one embodiment, the cell binding peptide binds to one or more of fibroblasts or stem cells. In one embodiment, the BMP binding peptide binds to one or more of BMP-2, BMP-4, BMP-6, or BMP-7. In one embodiment, the biopolymer is selected from the group consisting of a collagen, an injectable collagen, a fibrillar collagen, a Type I collagen, a bovine collagen, a recombinant collagen, an animal-derived collagen, a gelatin, an elastin, a keratin, a silk, a polysaccharide, an agarose, a dextran, a cellulose derivative, an oxidized cellulose, an oxidized regenerated cellulose, a carboxymethylcellulose, a hydroxypropylmethylcellulose, a chitosan, a chitin, a hyaluronic acid, and derivatives and combinations thereof.

In one embodiment, an implantable device for soft tissue repair is provided comprising a biopolymer having a covalently attached cell binding peptide, wherein the cell binding peptide comprises a sequence set forth in any one of SEQ ID NOs: 1-53, and wherein the implantable device comprising the biopolymer is selected from the group consisting of a gel, a hydrogel, an injectable material, an extracellular matrix, a decellularized tissue, a dermal matrix, a small intestinal submucosa (SIS), an acellular human dermis, an acellular porcine dermis, an acellular bovine dermis, an acellular myocardium, a cardiac patch, a heart valve, a surgical mesh, a skin graft, an injectable for dermal tissue augmentation, a dural graft, a graft for foot ulcer repair, a hernia repair graft, a graft for abdominal repair, a tendon wrap, a tendon augmentation graft, a graft for rotator cuff repair, a graft or mesh for breast reconstruction, a graft or mesh for pelvic floor reconstruction, a graft for medial collateral ligament repair, a graft for anterior cruciate ligament repair, a composite surgical mesh comprising a synthetic biopolymer and a biopolymer, and derivatives and combinations thereof.

In one embodiment, an implantable device for bone tissue repair is provided comprising a biopolymer having a covalently attached BMP binding polypeptide, wherein the BMP binding polypeptide comprises a sequence selected from the group consisting of SEQ ID NOs: 54-184, 189-192, and 198-203, and wherein the implantable device is a bone graft material further comprising a ceramic. In one embodiment, the ceramic is selected from the group consisting of calcium phosphate, calcium phosphate cement, biocompatible magnesium doped calcium phosphates, calcium carbonate, calcium sulfate, barium carbonate, barium sulfate, alphatricalcium phosphate (α-TCP), tricalcium phosphate (TCP), betatricalcium phosphate (β-TCP), hydroxyapatite (HA), biphasic calcium phosphate, biphasic composite between HA and β-TCP, alumina, zirconia, bioglass, biocompatible silicate glasses, biocompatible phosphate glasses, bone particles, and combinations and mixtures thereof. In one embodiment, the implantable bone graft material is in the form of a sponge, a granulized sponge, a granule, a putty, a strip, an injectable, or a formed piece. In general, the shape and size of the implantable graft material will preferably closely mimic the size and shape of the defect it is trying to repair. In one embodiment, the implantable device will be in the shape of a formed piece. In one embodiment, the implantable device is in an injectable form in which it will have a viscosity low enough to allow it to be injected into a defect site using a large bore syringe or a syringe/needle combination.

In one embodiment, a method is provided for tissue repair, comprising: delivering to a subject an implantable device for tissue repair, wherein the implantable device comprises a biopolymer having a covalently attached cell binding peptide and/or BMP binding peptide, wherein the cell binding peptide comprises a sequence set forth in any one of SEQ ID NOs: 1-53, wherein the BMP binding peptide comprises a sequence selected from the group consisting of SEQ ID NOs: 54-184, 189-192, and 198-203, and wherein the implantable device serves as a scaffold for tissue repair. In one embodiment, the cell binding peptide binds to one or more of fibroblasts or stem cells. In one embodiment, the BMP binding peptide binds to one or more of BMP-2, BMP-4, BMP-6, or BMP-7. In one embodiment, the biopolymer is selected from the group consisting of a collagen, an injectable collagen, a fibrillar collagen, a Type I collagen, a bovine collagen, a recombinant collagen, an animal-derived collagen, a gelatin, an elastin, a keratin, a silk, a polysaccharide, an agarose, a dextran, a cellulose derivative, an oxidized cellulose, an oxidized regenerated cellulose, a carboxymethylcellulose, a hydroxypropylmethylcellulose, a chitosan, a chitin, a hyaluronic acid, and derivatives and combinations thereof. In one embodiment, the tissue for repair is a soft tissue, the binding peptide is the cell binding peptide, and wherein the implantable device comprising the biopolymer is selected from the group consisting of a gel, a hydrogel, an injectable material, an extracellular matrix, a decellularized tissue, a dermal matrix, a small intestinal submucosa (SIS), an acellular human dermis, an acellular porcine dermis, an acellular bovine dermis, an acellular myocardium, a cardiac patch, a heart valve, a surgical mesh, a skin graft, an injectable for dermal tissue augmentation, a dural graft, a graft for foot ulcer repair, a hernia repair graft, a graft for abdominal repair, a tendon wrap, a tendon augmentation graft, a graft for rotator cuff repair, a graft or mesh for breast reconstruction, a graft or mesh for pelvic floor reconstruction, a graft for medial collateral ligament repair, a graft for anterior cruciate ligament repair, a composite surgical mesh comprising a synthetic biopolymer and a biopolymer, and derivatives and combinations thereof.

In one embodiment, the soft tissue for repair comprises any one or more of tendon, muscle, connective tissue, ligament, cardiac tissue, bladder tissue, or dermis. In one embodiment, the tissue for repair is a bone tissue, and the implantable device comprising the biopolymer is a bone graft material comprising a ceramic.

In one embodiment of the presently disclosed subject matter, a method is provided for capturing cells and/or BMP onto an implantable device for tissue repair, comprising: contacting a sample comprising cells and/or BMP with the implantable device, wherein the implantable device comprises a biopolymer having a covalently attached cell binding peptide and/or BMP binding peptide, wherein the cell binding peptide comprises a sequence set forth in any one of SEQ ID NOs: 1-53, wherein the BMP binding peptide comprises a sequence selected from the group consisting of SEQ ID NOs: 54-184, 189-192, and 198-203, and wherein the cells and/or BMP comprised in the sample are captured onto the implantable device through binding to the attached binding peptide. In one embodiment, the biopolymer is selected from the group consisting of a collagen, an injectable collagen, a fibrillar collagen, a Type I collagen, a bovine collagen, a recombinant collagen, an animal-derived collagen, a gelatin, an elastin, a keratin, a silk, a polysaccharide, an agarose, a dextran, a cellulose derivative, an oxidized cellulose, an oxidized regenerated cellulose, a carboxymethylcellulose, a hydroxypropylmethylcellulose, a chitosan, a chitin, a hyaluronic acid, and derivatives and combinations thereof. In one embodiment, the tissue for repair is a soft tissue comprising any one or more of tendon, muscle, connective tissue, ligament, cardiac tissue, bladder tissue, or dermis, and wherein the binding peptide is the cell binding peptide having binding to one or more of fibroblasts or stem cells. In one embodiment, the implantable device comprising the biopolymer is selected from the group consisting of a gel, a hydrogel, an injectable material, an extracellular matrix, a decellularized tissue, a dermal matrix, a small intestinal submucosa (SIS), an acellular human dermis, an acellular porcine dermis, an acellular bovine dermis, an acellular myocardium, a cardiac patch, a heart valve, a surgical mesh, a skin graft, an injectable for dermal tissue augmentation, a dural graft, a graft for foot ulcer repair, a hernia repair graft, a graft for abdominal repair, a tendon wrap, a tendon augmentation graft, a graft for rotator cuff repair, a graft or mesh for breast reconstruction, a graft or mesh for pelvic floor reconstruction, a graft for medial collateral ligament repair, a graft for anterior cruciate ligament repair, a composite surgical mesh comprising a synthetic biopolymer and a biopolymer, and derivatives and combinations thereof. In one embodiment, the tissue for repair is a bone tissue, and the implantable device comprising the biopolymer having covalently attached binding peptide is a bone graft material further comprising a ceramic. In one embodiment, the sample comprising cells comprises bone marrow, bone marrow aspirate (BMA), autologous or allogeneic stem cells, adipose tissue, stromal vascular fraction of adipose tissue, blood, blood products, platelets, platelet-rich plasma (PRP), umbilical cord blood, embryonic tissues, placenta, amniotic epithelial cells, tissue punch, omentum, or a homogeneous or heterogeneous population of cultured cells, or combinations or derivatives thereof. In one embodiment, the sample comprising BMP comprises autologous bone, allograft bone, xenograft bone, bone marrow, bone marrow aspirate (BMA), or recombinant BMP, or combinations or derivatives thereof.

In one embodiment of the presently disclosed subject matter, a method is provided for tissue repair, comprising: contacting a sample comprising cells and/or BMP with an implantable device comprising a biopolymer having a covalently attached cell binding peptide and/or BMP binding peptide, wherein the cell binding peptide comprises a sequence set forth in any one of SEQ ID NOs: 1-53, wherein the BMP binding peptide comprises a sequence selected from the group consisting of SEQ ID NOs: 54-184, 189-192, and 198-203, wherein the cells and/or BMP comprised in the sample are captured onto the implantable device through binding to the attached binding peptide; and delivering to a subject the implantable device for tissue repair comprising the captured cells and/or BMP, wherein the presence of the captured cells and/or BMP promotes tissue growth in the subject. In one embodiment, the subject is an animal or a human patient. In one embodiment, the biopolymer is selected from the group consisting of a collagen, an injectable collagen, a fibrillar collagen, a Type I collagen, a bovine collagen, a recombinant collagen, an animal-derived collagen, a gelatin, an elastin, a keratin, a silk, a polysaccharide, an agarose, a dextran, a cellulose derivative, an oxidized cellulose, an oxidized regenerated cellulose, a carboxymethylcellulose, a hydroxypropylmethylcellulose, a chitosan, a chitin, a hyaluronic acid, and derivatives and combinations thereof. In one embodiment, the tissue for repair is a soft tissue comprising any one or more of tendon, muscle, connective tissue, ligament, cardiac tissue, bladder tissue, or dermis, and wherein the binding peptide is the cell binding peptide having binding to one or more of fibroblasts or stem cells. In one embodiment, the implantable device comprising the biopolymer is selected from the group consisting of a gel, a hydrogel, an injectable material, an extracellular matrix, a decellularized tissue, a dermal matrix, a small intestinal submucosa (SIS), an acellular human dermis, an acellular porcine dermis, an acellular bovine dermis, an acellular myocardium, a cardiac patch, a heart valve, a surgical mesh, a skin graft, an injectable for dermal tissue augmentation, a dural graft, a graft for foot ulcer repair, a hernia repair graft, a graft for abdominal repair, a tendon wrap, a tendon augmentation graft, a graft for rotator cuff repair, a graft or mesh for breast reconstruction, a graft or mesh for pelvic floor reconstruction, a graft for medial collateral ligament repair, a graft for anterior cruciate ligament repair, a composite surgical mesh comprising a synthetic biopolymer and a biopolymer, and derivatives and combinations thereof. In one embodiment, the tissue for repair is a bone tissue, and the implantable device comprising the biopolymer having covalently attached binding peptide is a bone graft material further comprising a ceramic. In one embodiment, the sample comprising cells comprises bone marrow, bone marrow aspirate (BMA), autologous or allogeneic stem cells, adipose tissue, stromal vascular fraction of adipose tissue, blood, blood products, platelets, platelet-rich plasma (PRP), umbilical cord blood, embryonic tissues, placenta, amniotic epithelial cells, tissue punch, omentum, or a homogeneous or heterogeneous population of cultured cells, or combinations or derivatives thereof. In one embodiment, the sample comprising BMP comprises autologous bone, allograft bone, xenograft bone, bone marrow, bone marrow aspirate (BMA), or recombinant BMP, or combinations or derivatives thereof.

In one embodiment of the presently disclosed subject matter, a method is provided for capturing cells, comprising contacting a sample comprising cells with a cell binding peptide attached to a substrate, wherein the cell binding peptide comprises a sequence set forth in any one of SEQ ID NOs: 1-53, and wherein the cells comprised in the sample are captured onto the substrate through binding to the cell binding peptide. In one embodiment, the cell binding peptide binds to one or more of stem cells or fibroblasts and the sample comprising cells comprises one or more of stem cells or fibroblasts. In one embodiment, the sample comprising cells comprises bone marrow, bone marrow aspirate (BMA), autologous stem cells, allogeneic stem cells, adipose tissue, stromal vascular fraction of adipose tissue, blood, blood products, platelets, platelet-rich plasma (PRP), umbilical cord blood, embryonic tissues, placenta, amniotic epithelial cells, tissue punch, omentum, or a homogeneous or heterogeneous population of cultured cells, or combinations or derivatives thereof. In one embodiment, the substrate comprises metal, glass, plastic, synthetic matrix, silica gel, polymer, biopolymer, or derivatives or combinations thereof. In one embodiment, the substrate is in the form of beads, coated beads, gel, hydrogel, mesh, foam, foam metal, fibrous form, hollow fibers, or sheets. In one embodiment, the cells comprised in the sample are captured onto the substrate in the form of beads, and the beads having the captured cells are delivered to a subject. In one embodiment, the cell capture is performed by an adsorption column, an adsorption membrane, or a density centrifugation. In one embodiment, the cell binding peptide comprises one or more modifications to the peptide N-terminus, peptide C-terminus, or within the peptide amino acid sequence, to allow for attachment of the cell binding peptide to the substrate and/or release of the cell binding peptide from the substrate. In one embodiment, the method comprises a step of releasing the captured cells from the substrate, wherein the step of releasing the captured stem cells is one or more of a physical means, chemical means, enzymatic cleavage, or photoactivated means. For example, in one embodiment, the step of releasing the captured cells from the substrate is by a physical means comprising shaking or centrifugation. In one embodiment, the step of releasing the captured cells from the substrate is by a change in pH, a change in salt concentration, or a competitive inhibition binding with molecules that compete with the binding of the captured cells to the binding peptide(s). In one embodiment, the step of releasing the captured cells from the substrate is by cleaving the binding peptide, to which the captured cells are bound, from the substrate. Accordingly, in this embodiment the binding peptide can comprise one or more modifications to the peptide N-terminus, peptide C-terminus, or within the peptide amino acid sequence, to allow for its cleavage from the substrate. In one embodiment, the binding peptide comprises a disulfide bond and the peptide is cleaved from the substrate by addition of a reducing agent to cleave the disulfide bond such as, for example, dithiothreitol (DTT) or tris[2-carboxyethyl]phosphine (TCEP). In one embodiment, the binding peptide comprises an enzyme cleavage sequence and the peptide is cleaved from the substrate by addition of an enzyme that can cleave the sequence in the peptide such as, for example, the enzyme trypsin. In one embodiment, the modification to the binding peptide to allow release from the substrate comprises a disulfide group cleavable by addition of a reducing agent or comprises an amino acid sequence cleavable by addition of an enzyme. In one embodiment, the modification to the binding peptide to allow for release from the substrate comprises a photoactivatable or photoswitchable compound, such as, for example, the compound, 4-[(4-aminophenyl)azo]benzocarbonyl, that causes a change in the structure of the peptide. In one embodiment, the released cells are delivered to a human subject or an animal subject.

In one embodiment of the presently disclosed subject matter, a device is provided for chromatography comprising a cell binding peptide attached to a substrate, wherein the cell binding peptide comprises a sequence set forth in any one of SEQ ID NOs: 1-53.

In one embodiment of the presently disclosed subject matter, a method is provided for visualizing cells, comprising contacting a cell with a cell binding peptide comprising a visualization agent, wherein the cell binding peptide comprises a sequence set forth in any one of SEQ ID NOs: 1-53, and wherein the cell binding peptide binds to the cell to enable cell visualization. The visualization agent is any one of known compounds such as, for example, a visualization agent that is a fluorophore. In one embodiment, the visualization agent is a fluorophore such as, for example, Alexa 488- or Alex 594-labeled streptavidin from INVITROGEN, and the cells are detected by fluorescent microscopy. The visualization agent is attached to the binding peptide using known methods such as, for example, through a strepavidin-biotin interaction using a biotinylated binding peptide.

The following examples are provided to further describe certain aspects of the presently disclosed subject matter and are not intended to limit the scope of the presently disclosed subject matter.

### EXAMPLES

### Example 1

### Identification of Cell Binding Peptides by Phage Display

Peptides that bind adipose derived stem cells (ASCs) were identified by phage display biopanning. ASCs were culture amplified from processed liposuction aspirate (<3 passages) (ZEN-BIO, Research Triangle Park, N.C.). After biopanning, individual plaques were picked, grown overnight, and tested for ASC binding activity using whole cell ELISA according to the following procedure. Phage supernatant was incubated for 30 min at room temperature with ASC monolayers established by overnight culture in 96 well plates. Cells were washed four times with Dulbecco's Phosphate Buffered Saline (DPBS) containing 2% FBS, then incubated with 100 μl of horseradish peroxidase conjugated anti-M13 antibody. After 30 minutes at room temperature, cells were washed four times with DPBS containing 2% FBS. M13 antibody binding was detected by the addition of 3,3′,5,5′-Tetramethylbenzidine (TMB). Blue reaction product, indicating positive binding phage, was measured by absorbance at 370 nm in a microplate reader. For the phage displaying ASC binding activity, DNA sequences were analyzed and translated into peptide sequences using Vector NTI DNA Analysis software and are shown below in Table 1.

TABLE 1
Cell Binding Peptides

| SEQ | |
|---|---|
| ID | Amino acid sequence |
| NO: | (single letter code) |
| 1 | SSWHISHDSIGVLIR |
| 2 | SHCVLGHDGRSRCII |

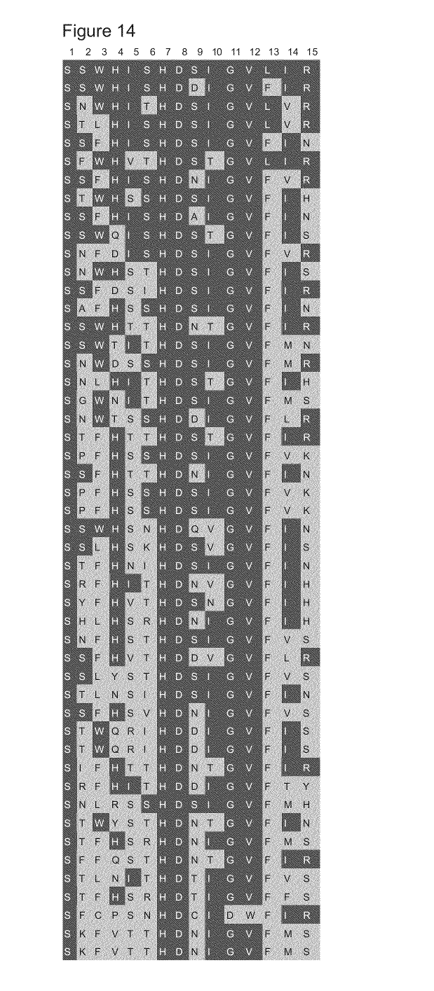

Mutagenesis of Cell Binding Peptide Sequence SEQ ID NO: 1. A focused phage display library was generated around the cell binding sequence SEQ ID NO: 1 with each nucleotide position varying in identity at a ratio of 70:10:10:10, with the original nucleotide being the dominant form. This is considered a form of “light” mutagenesis, retaining the majority of residue identities with a few amino acid identity changes. The construction of this “degenerate” phage library was performed according to the methods described in Kay et al., 1996. Three rounds of phage display biopanning were performed on ASCs to enrich for positive binding sequences. Phage showing positive binding to ASCs, identified by whole cell ELISA, were re-amplified, retested, and submitted for DNA sequencing to determine the insert amino acid sequences. The positive binders are shown below in Table 2 and are also shown in FIG. 14. In FIG. 14 the single letter amino acid sequence of SEQ ID NO: 1 is shown at the top in white letters with black shading. The phage that retained cell binding activity to adipose derived stem cells (ASCs) and fibroblasts are listed below SEQ ID NO: 1 with original amino acids in white with black shading. Amino acid substitutions are shown in black letters with white shading. The phage from the mutagenesis that did not exhibit cell binding activity are not shown.

TABLE 2
Cell Binding Peptides from Mutagenesis
Study of SEQ ID NO: 1.

| SEQ | |
|---|---|
| ID | Amino acid sequence |
| NO: | (single letter code) |
| 3 | SNWHITHDSIGVLVR |
| 4 | SFWHVTHDSTGVLIR |
| 5 | SNFDISHDSIGVFVR |
| 6 | SSWHISHDDIGVFIR |
| 7 | SRFHITHDNVGVFIH |
| 8 | STWYSTHDNTGVFIN |
| 9 | SSFHISHDNIGVFVR |
| 10 | SYFHVTHDSNGVFIH |
| 11 | SNLHITHDSTGVFIH |
| 12 | STFHSRHDNIGVFMS |
| 13 | SFFQSTHDNTGVFIR |
| 14 | SNWHSTHDSIGVFIS |
| 15 | SSFDSIHDSIGVFIR |
| 16 | SHLHSRHDNIGVFIH |
| 17 | SNFHSTHDSIGVFVS |
| 18 | STLNITHDTIGVFVS |
| 19 | STWHSSHDSIGVFIH |
| 20 | SSFHVTHDDVGVFLR |
| 21 | SAFHSSHDSIGVFIN |
| 22 | SSLYSTHDSIGVFVS |
| 23 | SSWHTTHDNTGVFIR |
| 24 | STLNSIHDSIGVFIN |
| 25 | STLHISHDSIGVLVR |
| 26 | SGWNITHDSIGVFMS |
| 27 | SSFHISHDAIGVFIN |
| 28 | SSWTITHDSIGVFMN |
| 29 | SNWTSSHDDIGVFLR |
| 30 | SSFHSVHDNIGVFVS |
| 31 | SSFHISHDSIGVFIN |
| 32 | STFHTTHDSTGVFIR |
| 33 | SPFHSSHDSIGVFVK |
| 34 | STWQRIHDDIGVFIS |
| 35 | STWQRIHDDIGVFIS |
| 36 | SSFHTTHDNIGVFIN |
| 37 | SPFHSSHDSIGVFVK |
| 38 | SPFHSSHDSIGVFVK |
| 39 | SIFHTTHDNTGVFIR |
| 40 | SKFVTTHDNIGVFMS |
| 41 | SKFVTTHDNIGVFMS |
| 42 | SRFHITHDDIGVFTY |
| 43 | SNWDSSHDSIGVFMR |
| 44 | STFHSRHDTIGVFFS |
| 45 | SSWHSNHDQVGVFIN |
| 46 | SSLHSKHDSVGVFIS |
| 47 | SNLRSSHDSIGVFMH |
| 48 | STFHNIHDSIGVFIN |
| 49 | SSWQISHDSTGVFIS |

Based on the foregoing results of the mutagenesis study of SEQ ID NO: 1, four cell binding sequence motifs were generated as follows: The first cell binding consensus motif (SEQ ID NO: 50) covers all of the SEQ ID NO: 1 variants shown in Table 2 and FIG. 14 that retain ASC binding properties (NOTE: Position 1 in FIG. 14 was not varied in the mutagenesis experiment and thus the consensus sequence corresponds to positions 2-15 in FIG. 14):
SEQ ID NO: 50: X₁-X₂-X₃-X₄-X₅-H-D-X₈-X₈-G-V-X₁₂-X₁₃-X₁₄
   wherein “X₁” is A, F, G, H, I, K, N, P, R, S, T, or Y; wherein “X₂” is F, L, or W; wherein “X₃” is D, H, N, Q, R, S, T, V, or Y; wherein “X₄” is I, N, R, S, T, or V; wherein “X₅” is I, K, N, R, S, T, or V; wherein “X₈” is A, D, N, Q, S, or T; wherein “X₉” is I, N, T, or V; wherein “X₁₂” is F, or L; wherein “X₁₃” is F, I, L, M, T, or V; and wherein “X₁₄” is H, K, N, R, S, or Y.

The second cell binding consensus sequence (SEQ ID NO: 51) is expanded relative to SEQ ID NO: 50 to additionally include conservative amino acid changes:
SEQ ID NO 51: X₁-X₂-X₃-X₄-X₅-H-X₇-X₈-X₈-G-X₁₁-X₁₂-X₁₃-X₁₄
   wherein “X₁” is A, F, G, H, I, K, N, P, R, S, T, V, W, or Y; wherein “X₂” is F, L, W, or Y; wherein “X₃” is D, H, I, K, N, Q, R, S, T, V, or Y; wherein “X₄” is I, K, L, N, Q, R, S, T, or V; wherein “X₅” is I, K, L, N, Q, R, S, T, or V: wherein “X₇” is D, or E; wherein “X₈” is A, D, E, N, Q, S, or T; wherein “X₉” is I, L, N, Q, S, T, or V; wherein “X₁₁” is V, I, L, or A; wherein “X₁₂” is F, L, or Y; wherein “X₁₃” is F, I, L, M, T, V, W, or Y; wherein “X₁₄” is H, K, N, Q, R, S, T, or Y. While the added conservative substitutions were not identified as actual cell binding phage sequences from the mutagenesis experiment listed in Table 2 and FIG. 14, these substitutions would be expected to have retained cell binding activity based on conservation of structure. Further, the 48 cell binding phage sequences that were analyzed for the mutagenesis experiment is not expected to be a sufficient number of sequences to give an exhautive list of every allowable change.

The third cell binding consensus sequence (SEQ ID NO: 52) is derived from the sequences shown in Table 2 and FIG. 14 that demonstrated the highest binding affinity for ASCs (specifically, SEQ ID NOs: 3, 4, 6, 9, 21, & 29 demonstrated the highest binding affinity):
SEQ ID NO 52: X₁-X₂-H-X₄-X₅-H-D-X₈-1-G-V-X₁₂-X₁₃-X₁₄
   wherein “X₁” is A, N, F, or S; wherein “X₂” is F, or W; wherein “X₄” is I, S, or V; wherein “X₅” is S, or T; wherein “X₈” is D, N, or, S; wherein “X₁₂” is F, or L; wherein “X₁₃” is I, L or V; wherein “X₁₄” is N or R.

The fourth cell binding consensus sequence (SEQ ID NO: 53) is an expansion of SEQ ID NO: 52 to further include amino acid substitutions that occurred in 10% or more of the sequences shown in Table 2 and FIG. 14 if not already present in SEQ ID NO: 52:
SEQ ID NO 53: X₁-X₂-H-X₄-X₅-H-D-X₈-X₉-G-V-X₁₂-X₁₃-X₁₄
   wherein “X₁” is A, N, F, S, or T; wherein “X₂” is F, L, or W; wherein “X₄” is I, S, T, or V; wherein “X₅” is S, or T; wherein “X₈” is D, N, or, S; wherein “X₉” is I or T wherein “X₁₂” is F, or L; wherein “X₁₃” is I, M, L or V; wherein “X₁₄” is H, N, R, or S.

### Example 2

### Generation of Synthetic Binding Peptides

Binding peptide sequences were synthesized using standard solid-phase peptide synthesis techniques on a SYMPHONY Peptide Synthesizer (PROTEIN TECHNOLOGIES, Tucson, Ariz.) using standard Fmoc chemistry (HBTU/HOBT activation, 20% piperidine in DMF for Fmoc removal). N-α-Fmoc-amino acids (with orthogonal side chain protecting groups; NOVABIOCHEM). After all residues were coupled, simultaneous cleavage and side chain deprotection was achieved by treatment with a trifluoroacetic acid (TFA) cocktail. Crude peptide was precipitated with cold diethyl ether and purified by high-performance liquid chromatography on a WATERS Analytical/Semi-preparative HPLC unit on VYDAC C18 silica column (preparative 10 μm, 250 mm×22 mm) using a linear gradient of water/acetonitrile containing 0.1% TFA. Homogeneity of the synthetic peptides was evaluated by analytical RP-HPLC (VYDAC C18 silica column, 10 μm, 250 mm×4.6 mm) and the identity of the peptides confirmed with MALDI-TOF-MS. Biotinylated peptides were generated similarly, with a GSSGK(biotin) sequence or other spacer group added to the C-terminus of the peptide.

### Example 3

### Cell Binding Peptide Specificity

Synthetic biotinylated cell binding peptides SEQ ID NOs: 1 and 2 were examined for their ability to specifically bind ASCs compared to a number of other cells types including bone marrow mesenchymal stem cells (MSCs), dermal fibroblasts, red blood cells, monocytes, lymphocytes granulocytes, and platelets. The cell binding peptides were biotinylated as described herein at Example 2. Cultured cells of each type were either purchased (dermal fibroblasts) or isolated from human bone marrow (MSCs) or human blood. Cells were first harvested with 2 mM EDTA in DPBS and resuspended at 10⁶/mL in PBS+2% fetal bovine serum (FBS). An aliquot of cells (50 pL) was incubated in 50 μL of peptide solution (25 μM in PBS+FBS) for 30 min at 4° C. Cells were then washed twice in PBS+2% FBS with 300×g centrifugation for 5 min between washes. Fluorescently-tagged neutravidin (Neutravidin-PE from INVITROGEN) was then added to the cells to label biotinylated peptide bound to cells. Neutravidin-PE was diluted 1:250 from stock and applied at 50 μL to cells. Cells were then washed in PBS+FBS, and acquired on a BD FACSARRAY. Peptide reactivity was then measured as percent positivity relative to Neutravidin-PE staining without the addition of biotinylated peptide. Cell binding peptides SEQ ID NOs: 1 and 2 were observed to have significant binding to human ASCs and dermal fibroblasts, but not to MSCs (data not shown).

### Example 4

### Capture of ASCs and Fibroblasts with Cell Binding Peptides Attached to a Substrate

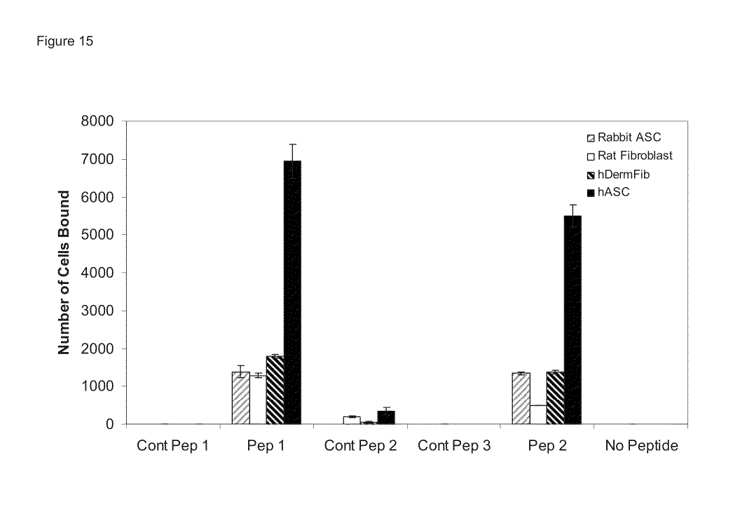

In this experiment, the ability of biotinylated cell binding peptides SEQ ID NOs: 1 and 2 to capture ASCs and fibroblasts from a single cell suspension onto a solid support was examined. The cell binding peptides were biotinylated as described herein at Example 2. The cell binding peptides and control peptides lacking cell binding activity were added to a 96 well plate containing immobilized streptavidin. After 30 min at room temperature, excess peptide was washed away with PBS+0.1% Tween 20, followed by 2 washes with PBS. Dilutions of single cell suspensions of either human ASCs (hASCs), rabbit ASCs, rat fibroblasts, or human dermal fibroblasts (hDermFib) were added to the peptide coated wells and incubated for 45 min at room temperature with gentle shaking. Wells were washed 3 times with DPBS+2% FBS. The number of attached cells was determined by measuring cellular ATP with CELLTITER GLO reagent from PROMEGA. The cell binding peptide SEQ ID NO: 1 was determined to capture human ASCs and to a lesser extent rabbit ASCs and rat and human fibroblasts (see FIG. 15). The cell binding peptide SEQ ID NO: 2 behaved similarly, except without exhibiting significant binding to rat fibroblasts (see FIG. 15).

### Example 5

### Covalent Attachment of Cell Binding Peptide to Collagen Substrate

Cell binding peptide SEQ ID NO: 1 was covalently attached to a collagen substrate using disulfide chemistry. HELISTAT collagen sponge (INTEGRA LIFE SCIENCES, Plainsboro, N.J.) was used as the collagen substrate. The cell binding peptide SEQ ID NO: 1 was modified at the carboxyl terminus with a PEG-10 spacer and a cystine residue.

Collagen Sponge Substrate Modification.

HELISTAT collagen (11 mg) was reacted with a solution of 2-iminothiolane hydrochloride (0.30 mg, 0.0020 mmol) and 6,6′-dithiodinicotinic acid (DTNA; 1.2 mg, 0.0040 mmol) in phosphate buffer (100 mM, pH 8.0) resulting in an intermediate activated for conjugation to a thiol. The general chemical scheme is shown in FIG. 4, except that the DTNA replaces the 4,4′-dithiodipyridine shown in FIG. 4. After washing the intermediate with phosphate buffer, it was reacted with cell binding peptide SEQ ID NO: 1 (with PEG-10-Cys modification; 2.1 mg) in 1×PBS (pH 7.4) and then washed with PBS and water. The peptide substitution level was determined by reduction of the final product with tris(2-carboxyethyl)phosphine (TCEP) followed by HPLC measurement of the released peptide (3.3 μmol/g collagen).

To assess the ability of the peptide-modified HELISTAT to bind cells, the following experiment was performed. Fifty thousand human dermal fibroblasts were added to 3 mm×3 mm coupons of HELISTAT modified with SEQ ID NO: 1 cell-binding peptide in 1 ml DPBS+2% FBS. Another group consisted of unmodified HELISTAT. After 45 min incubation with rotation, the matrices were washed 4 times with 1 ml DPBS+2% FBS to remove unbound cells and the number of attached cells was counted using CELLTITER-GLO (PROMEGA CORP, Madison, Wis.). Modification of the HELISTAT with the cell-binding peptide increased cell retention 9-fold relative to unmodified matrix (data not shown).

Soluble Collagen Substrate Modification.

In this experiment, cell binding peptide SEQ ID NO: 1 is covalently attached to a soluble collagen substrate using the 2-iminothiolane/DTNA chemistry described above for collagen sponge (the general chemical scheme is shown in FIG. 4, except that the DTNA replaces the 4,4-dithiodipyridine shown in the Figure). First, the collagen is reacted with 2-iminothiolane and DTNA, and then excess reagents are removed by dialysis. Second, the activated collagen intermediate is reacted with peptide followed by removal of excess peptide by dialysis. The level of peptide loading is determined by HPLC measurement of 6-MNA release during the reaction and reduction of final product with TCEP followed by HPLC measurement of the released peptide.

Fibroblast binding to soluble collagen modified with cell-binding peptide is assessed according to the following procedure. 96-well plates are coated with various amounts of unmodified or peptide-modified collagen over night at 4° C. Unbound collagen is removed and the plates are blocked. After washing, about 5,000 fibroblasts are added per well in serum-free medium for about 30 min at 37° C. The plates are washed, and bound cells are detected with CELLTITER-GLO (PROMEGA) using a luminometer.

Fibrillar Collagen Substrate Modification.

In this experiment, cell binding peptide SEQ ID NO: 1 is covalently attached to a fibrillar collagen substrate using the 2-iminothiolane/DTNA chemistry described above for collagen sponge (the general chemical scheme is shown in FIG. 4, except that the DTNA replaces the 4,4′-dithiodipyridine shown in the Figure). First, the collagen is reacted with 2-iminothiolane and DTNA and then washed to remove excess reagents. Second, the activated collagen intermediate is reacted with peptide followed by removal of excess peptide by washing. The level of peptide loading is determined by HPLC measurement of 6-MNA release during the reaction and reduction of final product with TCEP followed by HPLC measurement of the released peptide.

### Example 6

### Covalent Attachment of Cell Binding Peptide to Decellularized Tissue Substrates

Commercially Available Decellularized Tissues.

Cell binding peptide SEQ ID NO: 1 was covalently attached to a commercially available decellularized soft tissue matrix, XENFORM (fetal bovine dermis from TEI BIOSCIEMCES, Boston, Mass.) using the 2-iminothiolane/DTNA chemistry described above (the general chemical scheme is shown in FIG. 4, except that the DTNA replaces the 4,4′-dithiodipyridine shown in the Figure). Cell binding peptide SEQ ID NO: 1 was first modified with a spacer and cystine residue at the carboxyl terminus. The XENFORM matrix (11 mg) was reacted with a solution of 2-iminothiolane hydrochloride (0.028 mg, 0.00020 mmol) and 6,6′-dithiodinicotinic acid (DTNA; 0.12 mg, 0.00040 mmol) in phosphate buffer (100 mM, pH 8.0) resulting in an intermediate activated for conjugation to a thiol. After washing the intermediate with phosphate buffer, it was reacted with cell binding peptide SEQ ID NO: 1 (with PEG-10-Cys modification; 2.1 mg) in phosphate buffer (10 mM, pH 7.0) and then washed with phosphate buffer and water. The peptide substitution level was determined by reduction of the final product with tris(2-carboxyethyl)phosphine (TCEP) followed by HPLC measurement of the released peptide (1.1 μmol/g matrix).

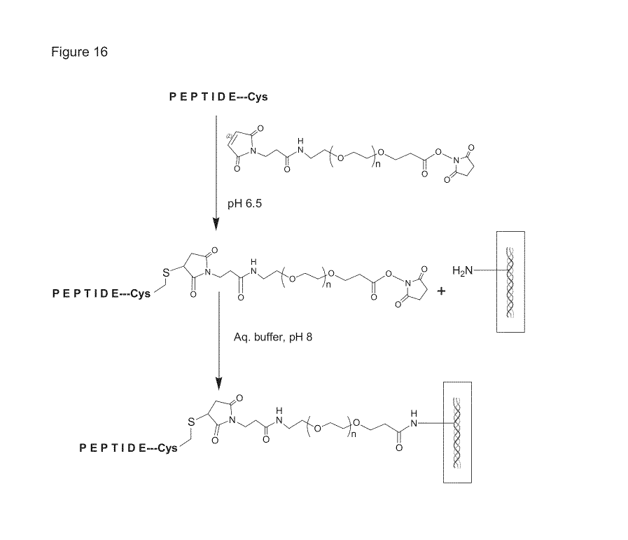

In another example, cell binding peptide SEQ ID NO: 1 was covalently attached to the commercially available decellularized soft tissue matrix, XENFORM, using the chemical scheme depicted in FIG. 16. First, the SEQ ID NO: 1 peptide having a C-terminal cysteine residue was dissolved in pH 6.5 buffer or anhydrous solvent (DMF). A hetero-bifunctional PEG-linker (PIERCE) was dissolved separately in anhydrous DMF (to minimize NHS hydrolysis) at 4° C. The reaction was initiated by adding the linker solution to the peptide solution at 4° C. and mixing at that temperature. The peptide was maintained in slight excess concentration over the PEG-linker. The reaction progress was monitored by HPLC (reaction was complete in about an hour). The excess solvents were removed by lyophilization. The peptide-PEG conjugate was used without further purification for attachment to pre-swollen XENFORM coupons. The peptide-PEG conjugate was dissolved in pH 8 PBS buffer and the coupons were transferred to peptide-PEG solution and gently shaken on a shaker apparatus at 4° C. for 48 h. The conjugate was washed thoroughly with water and freeze dried to obtain peptide modified matrix.

To assess the ability of the peptide-modified acellular matrices to bind cells, the following experiment was performed. Fifty thousand human dermal fibroblasts were added to the coupons of XENFORM modified with SEQ ID NO: 1 cell-binding peptide in 0.2 ml. Another group consisted of unmodified XENFORM. After 45 min incubation with agitation, the matrices were washed 3 times with 0.25 ml DPBS+2% FBS and the number of attached cells was counted using CELLTITER-GLO (PROMEGA CORP, Madison, Wis.). Modification of the acellular matrices with the cell-binding peptide increased cell retention 2-fold relative to unmodified matrix (data not shown).

### Example 7

### Cell Visualization with Cell Binding Peptides

In one example, cell binding peptides are labeled to enable detection of living cells by fluorescent microscopy. Cell binding peptides are first conjugated with Alexa 488 or Alex 594 labeled streptavidin (INVITROGEN). Biotinylated peptide (8 nmoles) is mixed with streptavidin-Alexafluor (2.3 nmoles) in DPBS for 1 h on ice. The peptide-streptavidin complex is added to a cell culture to be analyzed. After about a 15 min incubation at 37° C., unbound peptide-streptavidin complex is removed by washing with appropriate cell medium. Fresh growth medium is added back to the culture dish and live cell images are captured using DIC (differential interference contrast) and fluorescence using an inverted microscope.

### Example 8

### Identification of BMP Binding Peptides by Phage Display

Seven different phage display libraries were screened for binding to BMP. BMP2 (MEDTRONIC, INC.) was biotinylated with NHS-biotin (PIERCE) to produce a labeled protein with an average of one biotin per protein molecule. This protein was immobilized on streptavidin (SA) coated magnetic beads (DYNAL) and used as target for phage display. Selection was done in the presence of 0.5 M sodium chloride and 1% Tween-20. After 4 rounds of selection, individual phage isolates were tested for binding to biotinylated BMP-2 immobilized on SA coated plates. A conventional ELISA assay using anti-M13 phage antibody conjugated to HRP, followed by the addition of chromogenic agent THB. For the phage displaying BMP2 binding activity, DNA sequences were analyzed and translated into peptide sequences using Vector NTI DNA Analysis software and are shown below in Table 3.

TABLE 3
BMP Binding Phage Peptide Sequences

| SEQ | |
|---|---|
| ID | Amino acid sequence |
| NO: | (single letter code) |
| 54 | SIWDDFVGWSR |
| 55 | SIWDDWLGYSR |
| 56 | SIWDDWIGFSR |
| 57 | SIWDDFRASR |
| 58 | SIWDDYIGWTASGVGTSR |
| 59 | SIWDDYNRWLRGNSDISR |
| 60 | SIWDDYTRWGHKEASSSR |
| 61 | SIWHDFKSWKDNTPYHSR |
| 62 | SIWSDYLKWNAARGGASR |
| 63 | SIYDDFLNWKHGSVVPSR |
| 64 | SIYDDFRNWQISRVSDSR |
| 65 | SIYDDFVRWAMSSRADSR |
| 66 | SIYDNYLKWQERDRVTSR |
| 67 | SIWADYVRSSSASPLSR |

Peptide Synthesis.

BMP2 binding peptides were synthesized using standard solid-phase peptide synthesis techniques as described herein at Example 2. Biotinylated peptides were generated similarly, with a GSSGK(biotin) sequence or other spacer group added to the C-terminus of the peptide.

BMP Binding Activity.

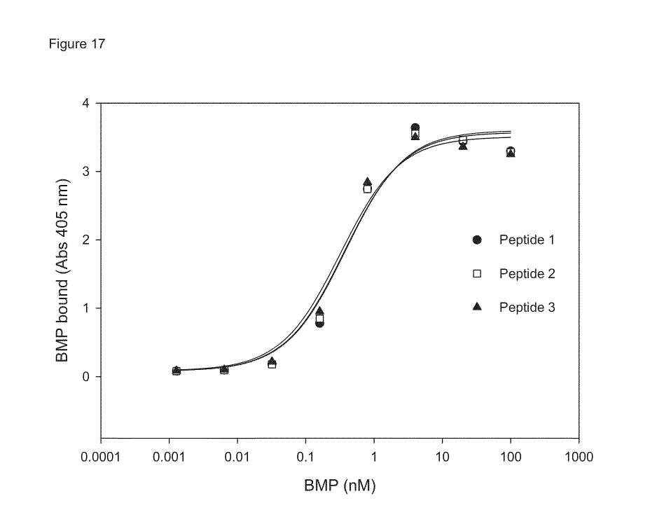

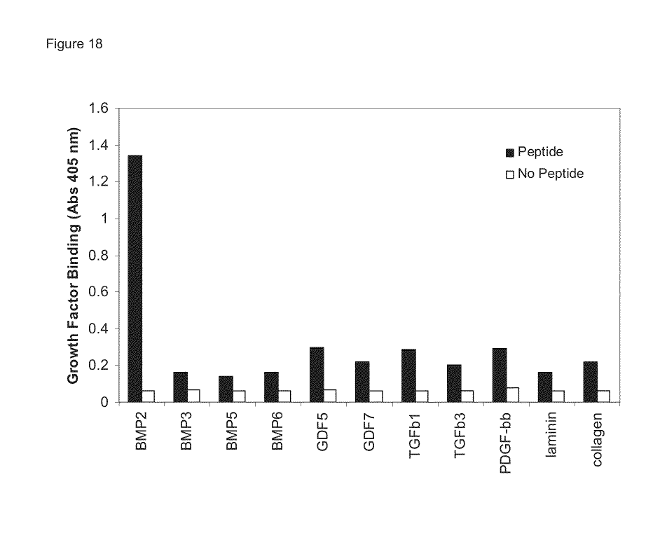

The relative binding affinity of synthetic BMP binding peptides SEQ ID NOs: 54-56 for BMP2 is shown in FIG. 17 (SEQ ID NO: 54 (Peptide 1); SEQ ID NO: 55 (Peptide 2); SEQ ID NO: 56 (Peptide 3)). Briefly, the results shown in FIG. 17 were generated by immobilizing biotinylated BMP binding peptides on streptavidin coated plates. Serial dilutions of 100 nM, 20 nM, 4 nM, 0.8 nM, 0.16 nM, 0.032 nM 0.006 nM, and 0.001 nM of BMP-2 were incubated with the immobilized BMP binding peptides for 60 minutes, washed 5 times, and bound BMP quantified using ELISA using mouse anti-BMP2 antibody and anti-mouse IgG-alkaline phosphatase conjugate. In addition, the specificity of BMP binding peptide binding SEQ ID NO: 54 to various members of the BMP family was performed in a similar manner by titrating BMP2, BMP3, BMP5, BMP6, GDF5, GDF7, TGFb1, TGFb3, and PDGF-BB (see FIG. 18).

A conservation of amino acid sequence was identified in the sequences for the BMP binding sequences shown in Table 3 above. To further analyze the contribution of the individual amino acids in the BMP binding sequences, SEQ ID NOs: 55 and 148 were chosen for scanning mutagenesis and truncation and experiments, respectively.

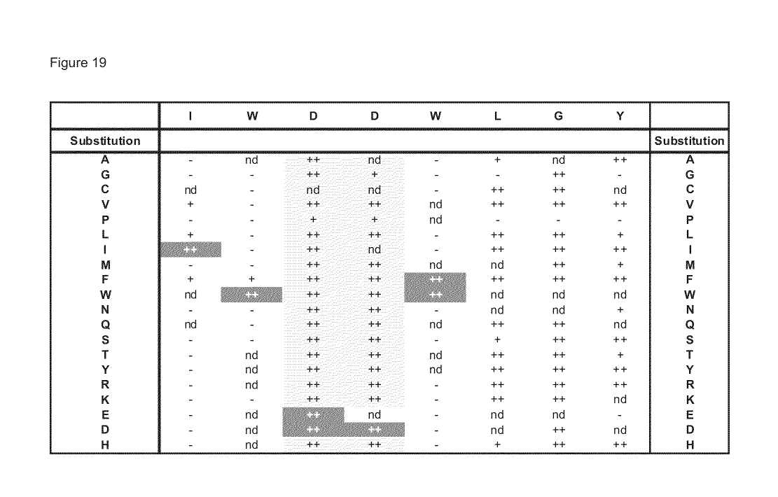

For the scanning mutagenesis analysis, eight M13 phage libraries were constructed from oligonucleotides that changed the codon for each shaded amino acid shown in the sequence SIWDDWLGYSR (SEQ ID NO: 55) to NNK (where N represents deoxynucleotides ACG or T, and K represents G or C). In this manner, phage libraries are generated that substitute each of these single amino acids within SEQ ID NO: 55 with all 20 naturally occurring amino acids. Individual M13 phage were picked randomly and tested for BMP2 binding and sequenced to determine the substituting amino acid. A table showing the variant peptide sequences generated is shown below (Table 4) and a table summarizing the effect of the amino acid substitutions on BMP2 binding activity is shown in FIG. 19.

TABLE 4
BMP2 Binding Variant Peptide Sequences
from Single Amino Acid Mutagenesis

| SEQ | |
|---|---|
| ID | Amino acid sequence |
| NO: | (single letter code) |
| 68 | SAWDDWLGYSR |
| 69 | SDWDDWLGYSR |
| 70 | SEWDDWLGYSR |
| 71 | SFWDDWLGYSR |
| 72 | SGWDDWLGYSR |
| 73 | SHWDDWMGYSR |
| 74 | SIWDDWLGYSR |
| 75 | SKWDDWLGYSR |
| 76 | SLWDDWLGYSR |
| 77 | SMWDDWLGYSR |
| 78 | SNWDDWLGYSR |
| 79 | SPWDDWLGYSR |
| 80 | SRWDDWLGYSR |
| 81 | SSWDDWLGYSR |
| 82 | STWDDWLGYSR |
| 83 | SVWDDWLGYSR |
| 84 | SYWDDWLGYSR |
| 85 | SICDDWLGYSR |
| 86 | SIFDDWLGYSR |
| 87 | SIGDDWLGYSR |
| 88 | SIIDDWLGYSR |
| 89 | SIKDDWLGYSR |
| 90 | SILDDWLGYSR |
| 91 | SIMDDWLGYSR |
| 92 | SINDDWLGYSR |
| 93 | SIPDDWLGYSR |
| 94 | SIQDDWLGYSR |
| 95 | SISDDWLGYSR |
| 96 | SIVDDWLGYSR |
| 97 | SIWADWLGYSR |
| 98 | SIWEDWLGYSR |
| 99 | SIWFDWLGYSR |
| 100 | SIWGDWLGYSR |
| 101 | SIWHDWLGYSR |
| 102 | SIWIDWLGYSR |
| 103 | SIWKDWLGYSR |
| 104 | SIWLDWLGYSR |
| 105 | SIWMDWLGYSR |
| 106 | SIWNDWLGYSR |
| 107 | SIWPDWLGYSR |
| 108 | SIWQDWLGYSR |
| 109 | SIWRDWLGYSR |
| 110 | SIWSDWLGYSR |
| 111 | SIWTDWLGYSR |
| 112 | SIWVDWLGYSR |
| 113 | SIWWDWLGYSR |
| 114 | SIWYDWLGYSR |
| 115 | SIWDFWLGYSR |
| 116 | SIWDGWLGYSR |
| 117 | SIWDHWLGYSR |
| 118 | SIWDKWLGYSR |
| 119 | SIWDLWLGYSR |
| 120 | SIWDMWLGYSR |
| 121 | SIWDNWLGYSR |
| 122 | SIWDPWLGYSR |
| 123 | SIWDQWLGYSR |
| 124 | SIWDRWLGYSR |
| 125 | SIWDSWLGYSR |
| 126 | SIWDTWLGYSR |
| 127 | SIWDVWLGYSR |
| 128 | SIWDWWLGYSR |
| 129 | SIWDYWLGYSR |
| 130 | SIWDDALGYSR |
| 131 | SIWDDCLGYSR |
| 132 | SIWDDDLGYSR |
| 133 | SIWDDELGYSR |
| 134 | SIWDDFLGYSR |
| 135 | SIWDDGLGYSR |
| 136 | SIWDDHLGYSR |
| 137 | SIWDDILGYSR |
| 138 | SIWDDKLGYSR |
| 139 | SIWDDLLGYSR |
| 140 | SIWDDNLGYSR |
| 141 | SIWDDRLGHSR |
| 142 | SIWDDSLGYSR |
| 143 | SIWDDWAGYSR |
| 144 | SIWDDWCGYSR |
| 145 | SIWDDWFGYSR |
| 146 | SIWDDWGGYSR |
| 147 | SIWDDWHGYSR |
| 148 | SIWDDWIGYSR |
| 149 | SIWDDWKGYSR |
| 150 | SIWDDWPGYSR |
| 151 | SIWDDWQGYSR |
| 152 | SIWDDWRGYSR |
| 153 | SIWDDWSGYSR |
| 154 | SIWDDWTGYSR |
| 155 | SIWDDWVGYSR |
| 156 | SIWDDWYGYSR |
| 157 | SIWDDWLCYSR |
| 158 | SIWDDWLDYSR |
| 159 | SIWDDWLFYSR |
| 160 | SIWDDWLHYSR |
| 161 | SIWDDWLIYSR |
| 162 | SIWDDWLKYSR |
| 163 | SIWDDWLLYSR |
| 164 | SIWDDWLMYSR |
| 165 | SIWDDWLPYSR |
| 166 | SIWDDWLQYSR |
| 167 | SIWDDWLRYSR |
| 168 | SIWDDWLSYSR |
| 169 | SIWDDWLVYSR |
| 170 | SIWDDWLYYSR |
| 171 | SIWDDWLGASR |
| 172 | SIWDDWLGESR |
| 173 | SIWDDWLGFSR |
| 174 | SIWDDWLGGSR |
| 175 | SIWDDWLGHSR |
| 176 | SIWDDWLGISR |
| 177 | SIWDDWLGLSR |
| 178 | SIWDDWLGMSR |
| 179 | SIWDDWLGNSR |
| 180 | SIWDDWLGPSR |
| 181 | SIWDDWLGRSR |
| 182 | SIWDDWLGSSR |
| 183 | SIWDDWLGTSR |
| 184 | SIWDDWLGVSR |

A summary of the mutagenesis analysis on BMP2 binding activity is shown in FIG. 19. The amino acid sequence of SEQ ID NO: 55 is shown at the top of FIG. 19 and the amino acid substitutions at each position are shown on both the far right and the far left of the Figure for convenience. The effects of the substitutions at each position on the BMP2 binding activity are depicted using the following symbols: (−) no binding; (+) moderate binding; (++) strong binding; (++) with stippled cells denotes strong, but non-specific binding; (++) with shaded cells denotes that only strong binders were found for that position; (nd) the amino acid substitution was not found in the phage tested.

For the truncation analysis, peptides with one or more N or C-terminal deletions of SEQ ID NO: 148 were synthesized and tested for BMP2 binding using an ELISA assay. The truncation peptides are shown in Table 5 below along with the relative BMP2 binding activity that was measured for each of the peptides according to the procedure described herein above. The relative BMP2 binding activity is depicted as follows: (−) no binding; (+) binding similar to that of full-length SEQ ID NO: 148 binding; (−/+) binding decreased relative to full-length SEQ ID NO: 148 binding.

TABLE 5
Truncation Analysis of SEQ ID NO: 148
on BMP2 Binding Activity.

| SEQ | | |
|---|---|---|
| ID | Peptide | BMP2 |
| NO: | Sequence | binding |
| 148 | SIWDDWIGYSR | + |
| 185 | .IWDDWIGYSR | − |
| 186 | ..WDDWIGYSR | − |
| 187 | ...DDWIGYSR | − |
| 188 | ....DWIGYSR | − |
| 189 | SIWDDWIGYS | + |
| 190 | SIWDDWIGY | + |
| 191 | SIWDDWIG | −/+ |
| 192 | SIWDDWI | −/+ |
| 193 | .IWDDWIGYS | − |
| 194 | .IWDDWIGY | − |
| 195 | ..WDDWIGY | − |
| 196 | ..WDDWIG | − |
| 197 | .IWDDWI | − |

Based on the foregoing results of the initial phage display to identify BMP binding peptides (Table 3), and the subsequent truncation (Table 5) and mutagenesis (FIG. 19) analyses of BMP binding peptides SEQ ID NO: 55 and SEQ ID NO: 148, respectively, six BMP binding sequence motifs were generated.

The first BMP binding consensus motif is based on the results of the scanning mutagenesis study of SEQ ID NO: 148 shown in FIG. 19:
S-I-W-D-D-X₆-X₇-X₈-Y (SEQ ID NO: 198)
   wherein “X₆” is F or W; wherein “X₇” is V or L; and wherein “X₈” is G or R.

The second BMP binding consensus motif is based on an alignment of the longest BMP binding sequences isolated by phage biopanning (Table 3) and is comprised of the amino acids that are most prevalent at each position:
S-I-X₃-D-D-X₆-X₇X₈-W (SEQ ID NO: 199)
   wherein “X₃” is for W or Y; wherein “X₆” is F, W, or Y; wherein “X₇” is I, L, or V; and wherein “X₈” is N or R.

The third BMP binding consensus motif is based on all the sequences that were isolated by phage biopanning (Table 3) and is comprised of the amino acids that were most prevalent at each position:
S-I-X₃-D-D-X₆-X₇-X₈-X₉ (SEQ ID NO: 200)
   wherein “X₃” is for W or Y; wherein “X₆” is F, W, or Y; wherein “X₇” is I, L, or V; wherein “X₈” is N or R; and wherein “X₉” is for F, W, or Y.

The fourth BMP binding consensus motif is based on all the sequences isolated by phage biopanning (Table 3). This consensus motif comprises amino acids that were prevalent at each position and also permits a conservative change from serine to threonine at position 1 (the Serine at position 1 was fixed in the phage biopanning experiment):
X₁-I-X₃-D-D-X₆-V-R-X₉ (SEQ ID NO: 201)
   wherein “X₁” is for S or T; wherein “X₃” is for W or Y; wherein “X₆” is F, W, or Y; and wherein “X₉” is for F, W, or Y.

The fifth BMP binding consensus motif is based on sequences isolated by phage biopanning (Table 3) and amino acids identified by codon scanning mutagenesis (FIG. 19). This sequence is comprised of amino acids that were prevalent at each position:
S-I-X₃-X₄-D X₆-X₇-X₈-X₉ (SEQ ID NO: 202)
   wherein “X₃” is for W or Y; wherein “X₄” is for D or E; wherein “X₆” is F, W, or Y; wherein “X₇” is I, L, or V; wherein “X₈” is N or R; and wherein “X₉” is for F, W, or Y.

The sixth BMP binding consensus motif is based on sequences isolated by phage biopanning (Table 3) and amino acids identified by codon scanning mutagenesis (FIG. 19). This sequence is comprised of amino acids that were most prevalent at each position and permits any aromatic amino acid at positions 3, 6 and 9:
S-I-X₃-X₄-D X₆X₇-X₈-X₉ (SEQ ID NO: 203)
   wherein “X₃” is for F, W or Y; wherein “X₄” is for D or E; wherein “X₆” is F, W, or Y; wherein “X₇” is L, or V; wherein “X₈” is N or R; and wherein “X₉” is for F, W, or Y.

The foregoing description of the specific embodiments of the presently disclosed subject matter has been described in detail for purposes of illustration. In view of the descriptions and illustrations, others skilled in the art can, by applying current knowledge, readily modify and/or adapt the presently disclosed subject matter for various applications without departing from the basic concept of the presently disclosed subject matter; and thus, such modifications and/or adaptations are intended to be within the meaning and scope of the appended claims.

What is claimed is:

1. A BMP binding peptide comprising a sequence selected from the group consisting of SEQ ID NOs: 198, 199, 200, 201, and 203.

2. The BMP binding peptide of claim 1, wherein the peptide comprises up to 30 amino acids.

3. The BMP binding peptide of claim 1, wherein the peptide comprises SEQ ID NO: 55.

4. The BMP binding peptide of claim 1, wherein the BMP binding peptide comprises one or more modifications to the peptide N-terminus, peptide C-terminus, or within the peptide amino acid sequence, wherein the modification is selected from the group consisting of aldehyde group, hydroxyl group, thiol group, amino group, amino acids, lysine, cysteine, acetyl group, biopolymers, synthetic biopolymers, polyethers, poly(ethylene glycol) (“PEG”), an 11 unit polyethylene glycol (“PEG10”), and a 1 unit polyethylene glycol (“mini-PEG” or “MP”), and combinations thereof.

5. The BMP binding peptide of claim 1, wherein the BMP binding peptide binds to one or more of BMP-2, BMP-4, BMP-6, or BMP-7.

6. An implantable device for tissue repair comprising a biopolymer having a covalently attached BMP binding peptide, wherein the BMP binding peptide comprises a sequence selected from the group consisting of SEQ ID NOs: 198, 199, 200, 201, and 203.

7. The implantable device of claim 6, wherein the BMP binding peptide comprises one or more modifications to the peptide N-terminus, peptide C-terminus, or within the peptide amino acid sequence, wherein the modification is selected from the group consisting of aldehyde group, hydroxyl group, thiol group, amino group, amino acids, lysine, cysteine, acetyl group, biopolymers, synthetic biopolymers, polyethers, poly(ethylene glycol) (“PEG”), an 11 unit polyethylene glycol (“PEG10”), and a 1 unit polyethylene glycol (“mini-PEG” or “MP”), and combinations thereof.

8. The implantable device of claim 6, wherein the BMP binding peptide is attached to the biopolymer with or without a spacer.

9. The implantable device of claim 6, wherein the BMP binding peptide binds to one or more of BMP-2, BMP-4, BMP-6, or BMP-7.

10. The implantable device of claim 6, wherein the BMP binding peptide comprises up to 30 amino acids.

11. The implantable device of claim 6, wherein the biopolymer is selected from the group consisting of a collagen, an injectable collagen, a fibrillar collagen, a Type I collagen, a bovine collagen, a recombinant collagen, an animal-derived collagen, a gelatin, an elastin, a keratin, a silk, a polysaccharide, an agarose, a dextran, a cellulose derivative, an oxidized cellulose, an oxidized regenerated cellulose, a carboxymethylcellulose, a hydroxypropylmethylcellulose, a chitosan, a chitin, a hyaluronic acid, and derivatives and combinations thereof.

12. The implantable device of claim 6, further comprising a ceramic for bone tissue repair.

13. The implantable device of claim 12, wherein the ceramic is selected from the group consisting of calcium phosphate, calcium phosphate cement, biocompatible magnesium doped calcium phosphates, calcium carbonate, calcium sulfate, barium carbonate, barium sulfate, alphatricalcium phosphate (α-TCP), tricalcium phosphate (TCP), betatricalcium phosphate (β-TCP), hydroxyapatite (HA), biphasic calcium phosphate, biphasic composite between HA and β-TCP, alumina, zirconia, bioglass, biocompatible silicate glasses, biocompatible phosphate glasses, bone particles, and combinations and mixtures thereof.

14. The implantable device of claim 13, in the form of a sponge, a granulized sponge, a granule, a putty, a strip, an injectable, or a formed piece.

15. A method for binding BMP, the method comprising contacting a sample having BMP with a BMP binding peptide, wherein the BMP binding peptide comprises a sequence selected from the group consisting of SEQ ID NOs: 198, 199, 200, 201, and 203.

16. The method of claim 15, wherein the sample having BMP comprises autologous bone, allograft bone, xenograft bone, bone marrow, bone marrow aspirate (BMA), recombinant BMP, combinations thereof, or derivatives thereof.

17. The method of claim 15, wherein the BMP binding peptide comprises one or more modifications to the peptide N-terminus, peptide C-terminus, or within the peptide amino acid sequence, wherein the modification is selected from the group consisting of aldehyde group, hydroxyl group, thiol group, amino group, amino acids, lysine, cysteine, acetyl group, biopolymers, synthetic biopolymers, polyethers, poly(ethylene glycol) (“PEG”), an 11 unit polyethylene glycol (“PEG10”), and a 1 unit polyethylene glycol (“mini-PEG” or “MP”), and combinations thereof.

18. The method of claim 15, wherein the BMP binding peptide binds to one or more of BMP-2, BMP-4, BMP-6, or BMP-7.

19. The method of claim 15, wherein the BMP binding peptide comprises up to 30 amino acids.

20. The method of claim 15, wherein the BMP binding peptide comprises SEQ ID NO: 55.
